(19) United States
(12) Patent Application Publication
(10) Pub. No.: US 2017/0266353 A1
(43) Pub. Date: Sep. 21, 2017

(54) BIOADHESIVE COMPOUNDS AND METHODS OF SYNTHESIS AND USE

(71) Applicant: DSM IP ASSETS, B.V., Heerlen (NL)

(72) Inventors: John L MURPHY, Exton, PA (US); Christopher P RADANO, Exton, PA (US); Jeffrey L. DALSIN, Exton, PA (US); Justin T. KOEPSEL, Exton, PA (US)

(21) Appl. No.: 15/309,695

(22) PCT Filed: May 13, 2015
(86) PCT No.: PCT/US2015/030574

§ 371 Date: Nov. 8, 2016

Related U.S. Application Data
(60) Provisional application No. 61/992,645, filed on May 13, 2014.

(51) Int. Cl. A61L 31/10; A61L 29/08; A61L 29/10; A61L 31/08

(52) U.S. Cl. CPC A61L 31/10; A61L 31/088; A61L 29/085; A61L 29/106; A61L 2300/104; A61L 2300/404; A61L 2400/18

(57) ABSTRACT

Synthesis methods for creating polymeric compounds comprising phenyl derivatives (PD), or PDp i.e., polymers modified with PD, with desired surface active effects are described. The polymer backbone of PDp has structural or performance features that can be tailored to control physical properties of PDp, allowing it to be useful for different applications i.e., tissue adhesives or sealants, adhesion promoting coatings, and antifouling coatings.

### REFERENCE TO FEDERAL FUNDING

This invention was made with United States government support under grant number #R44DK080547 awarded by NIH NIDDK. The United States government has certain rights in the invention.

### BACKGROUND

Marine mussels are known for their ability to bind tenaciously to such varied surfaces as rocks, pilings, and ship hulls in a wet, turbulent, and saline environment. These marine organisms secrete adhesive proteins as liquids that rapidly harden to form adhesive plaques, all under water, allowing them to attach themselves to various surfaces. The water-resistant adhesive characteristics of mussel adhesive proteins (MAPs) are believed to be due to the presence of 3,4-dihydroxyphenylalanine (DOPA), which is also responsible for both interfacial adhesion and rapid hardening.

There have been numerous attempts to engineer compounds that mimic the adhesive proteins secreted by marine mussels. These methods include the extraction of natural MAPs, the use of recombinant DNA technologies to create adhesive proteins, and synthesis of DOPA-containing peptides using both solid-phase and solution-phase methods. Although these MAP-mimetic adhesives demonstrate strong adhesion to various surfaces, their adhesive formulations utilize peptide backbones, which can be costly to mass-produce and have limited physical properties. Messersmith and colleagues have recently developed a series of DOPA-modified synthetic polymeric gels that demonstrate strong water-resistant adhesion. The same research group has also prepared coatings that can repel protein and cellular adsorption by chemically coupling a MAP-mimetic peptides to antifouling synthetic polymers.

In an alternative approach, other phenolic alternatives may also serve to bind to a surface under conditions similar to those in which the DOPA has previously been utilized. These phenolic derivatives, such as catechol, guaiacol and syringol derivatives, are naturally occurring compounds with a variety of functions. Catechol moieties are often associated with mussel adhesive proteins (MAPs) which utilize this derivative to form tenacious bonds in aqueous solutions. Alternatively, guaiacol and syringol derivatives are often associated with plants, and form the structural components of lignins. These structural components are formed through the oxidative crosslinking of the phenolic group to form polymeric structures. It was found this oxidative process also forms covalent bonds between amines and thiols on tissue surfaces.

The approach of combining synthetic polymers with DOPA and its dihydroxyphenyl derivatives (DHPD) to form DHPD-modified adhesive polymers (DHPp), or alternatively combining synthetic polymers with phenolic derivatives (PDs) to form PD modified polymers (PDp) may have numerous applications in clinical, dental, and industrial arenas. The general structure of PDp is shown in FIG. 1. PDp can impart strong water-resistant adhesion, as well as rapid and controllable intermolecular curing of the adhesive polymers. Different synthetic polymers can be used to control other physical properties such as but not limited to biocompatibility, solubility, biodegradability, self-assembling ability, chemical architecture, stimulus-response ability, branching, and molecular weight. Thus these molecules can be tailored to a particular use by varying the polymer portion of the compound. Specifically, the adhesive polymers described here not only can be designed to promote adhesion between two dissimilar surfaces, they can also be designed to prevent adhesion of undesirable particles (i.e. cells, proteins bacteria, etc).

For example, bacterial attachment and biofilm formation are serious problems associated with the use of urinary stents and catheters as they often lead to chronic infections that cannot be resolved without removing the device. Although numerous strategies have been employed to prevent these events including the alteration of device surface properties, the application of anti-attachment and antibacterial coatings, host dietary and urinary modification, and the use of therapeutic antibiotics, no one approach has yet proved completely effective. This is largely due to three important factors, namely various bacterial attachment and antimicrobial resistance strategies, surface masking by host urinary and bacterial constituents, and biofilm formation.

While the urinary tract has multiple anti-infective strategies for dealing with invading microorganisms, the presence of a foreign stent or catheter provides a novel, non-host surface to which they can attach and form a biofilm. This is supported by studies highlighting the ability of normally non-uropathogenic microorganisms to readily cause device-associated urinary tract infections. Ultimately, for a device to be clinically successful it must not only resist bacterial attachment but that of urinary constituents as well. Such a device would better allow the host immune system to respond to invading organisms and eradicate them from the urinary tract. For example, bacterial attachment and subsequent infection and encrustation of uropathogenic E. coli (UPEC) cystitis is a serious condition associated with biofouling. Infections with E. coli comprise over half of all urinary tract device-associated infections, making it the most prevalent pathogen in such episodes. The present invention also surprisingly provides unique antifouling coatings/constructs that could be used anywhere that a reduction in bacterial attachment is desired, for example, dental unit waterlines, implantable orthopedic devices, cardiovascular devices, wound dressings, percutaneous devices, surgical instruments, marine applications, food preparation surfaces and utensils. Compositions, methods, kits and systems of the present invention find use, for example, in medical diagnostics and therapeutics including but not limited to preparation of nonfouling surfaces for biosensors, cardiovascular implants, catheters; lubricious coatings on catheters, needles, and other percutaneous devices; medical tubing (dialysis); implantable electronic devices (MEMS); corrosion resistant coatings on medical grade metal alloys (surface adsorbed catechols are unknown to enhance corrosion resistance of metals); stabilization of particles of diagnostics and therapy, and nonmedical Applications, including but not limited to: corrosion resistant coatings (surface adsorbed catechols and polyphenols are known to enhance corrosion resistance of metals, and polyphenol polymers that are currently used as corrosion resistant coatings); antifouling coatings on consumer goods (for example, sunglasses, etc.); antifouling coatings on electronic devices (MEMS, etc.); antifouling/anti-icing coatings on aircraft watercraft and the like.

Additionally, inexpensive starting materials are used for the syntheses, which allow the subsequent adhesive polymers to be prepared inexpensively and in large quantities for commercialization. Furthermore, starting materials of known biocompatibility can be used to formulate these polymers, which makes them suitable for clinical applications.

New approaches to creating adhesive polymers modified with multiple PD are described herein. Different synthetic methods were used to combine the adhesive moiety, PD, with various biocompatible, synthetic compounds to create a library of adhesive polymers that can be designed for a desired application. These multi-PD polymers were tested for their potential as tissue adhesives, coatings for promoting adhesion, and coatings for adhesion prevention.

### SUMMARY OF THE INVENTION

Briefly, in one aspect, the present invention is a polymer or copolymer comprising a polymer backbone (pB) having attached, generally pendant, phenyl derivatives (PDs) to form a PD-modified polymer (PDp) having: 1) a variable concentration, distribution, or number of PD moieties, which account for about 1 to about 100% by weight PDp, preferably about 1-75% by weight in PDp, 2) a total molecular weight between 1,000 and 5,000,000 Da, and 3) a pB with variable physical properties.

In an embodiment of this aspect of the invention, PD comprises from about 1 to about 65 weight percent of PDp; in another embodiment PD comprises from about 2 to about 55 weight percent PDp, and in still another embodiment, PD comprises at least about 3 to about 50 weight percent PDp.

In an embodiment of this aspect of the invention, PDp has a total molecular weight in the range of about 3,000 to about 1,000,000 most preferably about 5,000 to about 500,000 Da.

More particularly, this present invention comprises a pB with pendant PD providing a PDp generally of the structure (I):

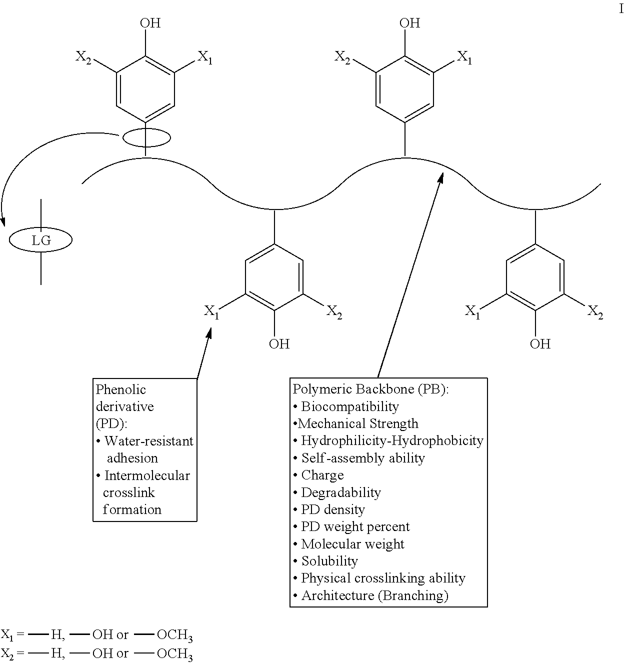

wherein LG is an optional linking group and pB indicates the polymer backbone.

In PDp, the PD imparts: 1) the ability to bind to or adhere to a dissimilar substrate, surface, compound, or particle, both organic and inorganic, in an aqueous, humid, or non-aqueous environment, and 2) the ability to form irreversible (covalent bond) or reversible (hydrogen bond, electron π-π interaction, dipole-dipole interactions, electrostatic interactions, etc.) chemical crosslinks either with other PD, other functional groups (i.e. amine, thiol, hydroxyl, or carboxyl groups), or other reactive groups.

Additionally, the composition and chemical structure of the polymer backbone can be varied to control 1) the PD weight percent, 2) the molecular weight of the PDp, and 3) the physical properties of PDp (solubility, hydrophilicity-hydrophobicity, physical crosslinking ability, self-assembly ability, architecture, charge, degradability, among others) for a desired application.

In a further aspect, the present invention is a polymer or copolymer comprising a pB having a controllable and variable number, concentration, or distribution of pendant PDs relative to the molecular weight monomers, prepolymers, or oligomers having variable chemical compositions or containing pendant groups or moieties distributed along and between the PD pendant moieties (and in the PB) as shown in structural formula (II):

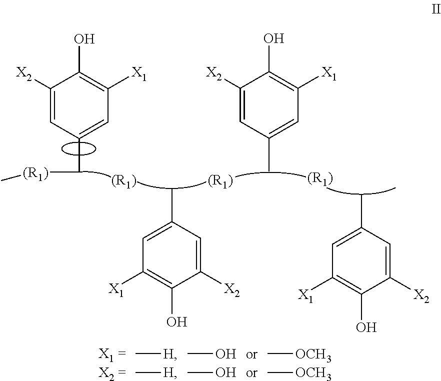

wherein R₁ is a monomer, prepolymer, or oligomer linked or polymerized to form pB. The polymer backbone has structural or performance features or characteristics designed or introduced into it by means of the “in-line” or backbone linkages, R₁. In-line or backbone linkages or linking groups can be introduced to control or modify all of the polymer characteristics shown in the right box of Formula (I). Examples of such backbone linkages include but are not limited to amide, ester, urethane, urea, carbonate, or carbon-carbon linkages or the combination thereof

Generally, PD can be illustrated as structural formula (III):

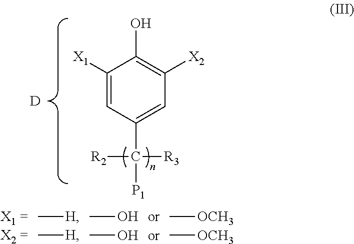

wherein n may be an integer from 1-6, R₂ and R₃ may be the same or different and are independently selected from the group consisting of hydrogen, saturated and unsaturated, branched and unbranched, substituted and unsubstituted C₁-₄ hydrocarbon;

P₁ is separately and independently selected from the group consisting of —NH₂, —COOH, —OH, —SH,

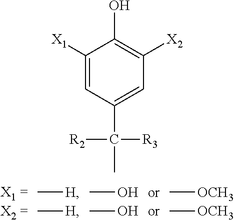

wherein R₂ and R₃ are defined above,

a halogen,

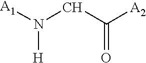

wherein A₁ and A₂ are separately and independently selected from the group consisting of H, a protecting group, substantially poly(alkyleneoxide),

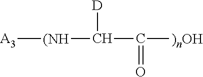

wherein n=1-3

and A₃ is

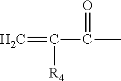

R₄ is H, C₁₆ lower alkyl, or

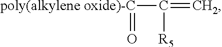

R₅ is defined the same as R₂ or R₃, above, and D is indicated in Formula (III).

In one aspect the poly(alkylene oxide) has the structure

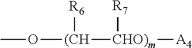

wherein R₆ and R₇ are separately and independently —H, or —CH₃ and m has a value in the range of 1-250, A₄ is —NH₂, —COOH, —OH, —SH, —H or a protecting group.

In an alternative embodiment, PD is

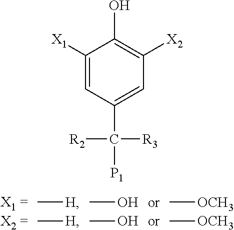

R₂, R₃, and P₁ being defined as above.

In another alternative embodiment PD is of the structure:

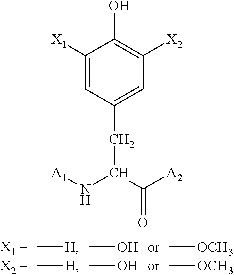

wherein A₂ is —OH and A₁ is substantially poly(alkylene oxide) of the structure

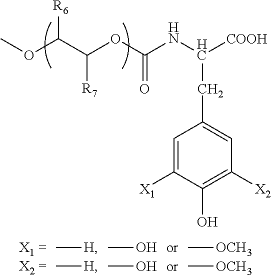

wherein R₆, R₇ and m being defined as above. Generally speaking, the poly(alkylene oxide) is a block copolymer of ethylene oxide and propylene oxide.

A method of this invention involves adhering substrates to one another comprising the steps of providing PD of the structure:

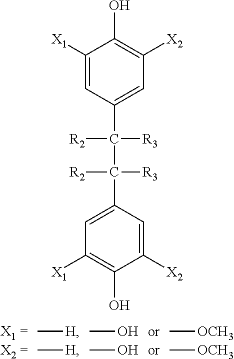

wherein R₂ and R₃ are defined as above; applying the PD of the above structure to one or the other or both of the substrates to be adhered; contacting the substrates to be adhered with the PD of the above structure there between to adhere the substrates to each other, and optionally repositioning the substrates relative to each other by separating the substrates and recontacting them to each other with the PD of the above structure there between.

In another method, R₂ and R₃ are hydrogen.

In another embodiment, the PD is:

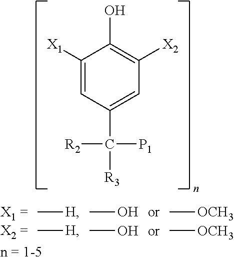

wherein P₁, R₂, and R₃ are defined above, and n ranges between 1 and about 5. In one practice, R₂ and R₃ are hydrogen and P₁ is, itself. Another embodiment which provides for PD in a practice of the present invention is 3-methoxytyramine, tyramine, 3,5-dimethoxy-4-hydroxyphenethylamine, vanillylamine, 4-hydroxybenzylamine, 4-hydroxy-3-methoxy-L-phenylalanine, N-benzoyl-4-hydroxy-3-methoxyphenylalanine, dopamine, 3,4-dihydroxyphenylalanine

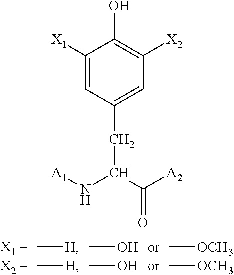

wherein A₁ and A₂ are defined above.

In yet another aspect of the present invention, PD has a general chemical structure

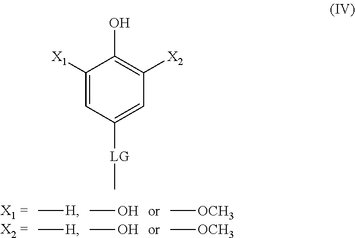

formula (IV):

wherein LG is a linking group that attaches PD to PB and is further defined below; LG is chosen from oligomers of substantially poly(alkylene oxide), acrylate, methacrylate, vinyl groups, and their derivatives, or having chemical structure formula (V):

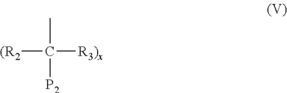

wherein R₂ and R₃ are defined above; x is a value between zero and four;

P₂ is selected from the group consisting of —NH₂, —COOH, —OH, —SH, a single bond, halogen,

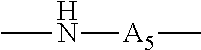

wherein A₅ is selected from the group consisting of —H, —C, a single bond, a protecting group, substantially alkyl, poly(alkylene oxide), peptidal, glycosidic, acrylated, methacrylated, or the same as A₁ and A₂;

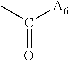

wherein A₆ is selected from the group of —OH, —NH—, in addition to the definition of A₁;

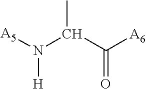

wherein A₅ and A₆ are defined above.

In one embodiment, the chemical structure of PD is:

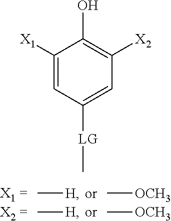

wherein LG is defined above.

In another embodiment, PD is:

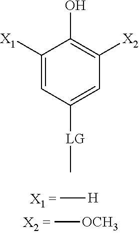

wherein LG is defined above.

An alternate form of PD is:

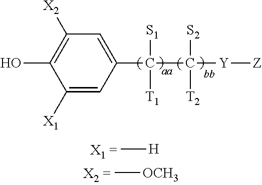

wherein Y is —NH₂, —COOH, —SH, —OH;

Z is optional and is the reaction product of an acrylate, methacrylate or other vinyl group

each S₁, independently, is H, NH₂, OH, or COOH;

each T₁, independently, is H, NH₂, OH, or COOH;

each S₂, independently, is H, NH₂, OH, or COOH;

each T₂, independently, is H, NH₂, OH, or COOH;

Optionally provided that when one of the combinations of S₁ and S₂, T₁ and T₂, Sᵢ and T₂ or T₁ and S₂ are absent, then a double bond is formed between the Cₐₐ and C_{bb}, further provided that aa and bb are each at least 1 to form the double bond when present.

In an embodiment, the PD may be chosen from 3,4-dihydroxyhydrocinnamic acid, 3,4-dihydroxyphenylalanine, dopamine, 3-methoxytyramine, tyramine, 3,5-dimethoxy-4-hydroxyphenethylamine, vanillylamine, 4-hydroxybenzylamine, 4-hydroxy-3-methoxy-L-phenylalanine, N-benzoyl-4-hydroxy-3-methoxyphenylalanine, ferulic acid, caffeic acid, vanillic acid, syringic acid, sinapic acid, hydroferulic acid, homovanillic acid, 3,4-dihydroxybenzoic acid, gallic acid, 4-hydroxybenzoic acid, isoferulic acid, p-coumaric acid, 4-hydroxyphenylacetic acid, 3,4-dihydroxyphenylacetic acid. Examples of further derivatized forms of PD include PD with protecting group(s), PD bound to metal ion on the hydroxyl group(s), or PD modified with acrylate, methacrylate, substantially poly(alkylene oxide), peptide, glycosidic, or oligomer containing PD and a combination thereof.

The composition and physical properties of pB are varied by the physical properties of, ratio of, composition, or combination of monomers or prepolymers used to construct said pB. In an embodiment, pB is constructed by polymerization, chain extension, linking, crosslinking or reaction of a single or more than one type of monomer or prepolymer.

pB is preferably a) linear or branched, b) mono-, bi-, tri-, or multifunctional to achieve a pB with linear, branched , hyperbranched, or brush architecture.

pB is preferably hydrophilic, hydrophobic or amphiphilic to achieve the desired solubility, stiffness, physical crosslinking ability, or self-assembly characteristics.

pB is preferably neutral, positively or negatively charged, or a combination thereof to achieve a neutral, charged, or zwitterionic pB.

pB is preferably polyether, polyester, polyamide, polyurethane, polycarbonate, or polyacrylate among many others and the combination thereof.

pB can be constructed of different linkages, but is preferably comprised of acrylate, carbon-carbon, ether, amide, urea, urethane, ester, or carbonate linkages or a combination thereof to achieve the desired rate of degradation or chemical stability.

pB of desired physical properties can be selected from prefabricated functionalized polymers or FP, a pB that contain functional groups (i.e. amine, hydroxyl, thiol, carboxyl, vinyl group, etc.) that can be modified with PD to form PDp.

pB may be a biopolymer. A biopolymer is obtained from natural sources, such as, but not limited to alginate, heparin, collagen, gelatin, polypeptides, nucleic acids, sugars, silk, or a combination of these.

The actual method of linking the monomer or prepolymer to form a pB will result in the formation of amide, ester, urethane, urea, carbonate, or carbon-carbon linkages or the combination of these linkages, and the stability of the pB is dependent on the stability of these linkages.

The molecular weight of monomer or prepolymer can vary between about 50 and 20,000 Da but is preferentially between 60 and 10,000 Da.

The monomer or prepolymer is preferably a single compound or repeating monomer units of a single-, bi-, tri-, or multi-block structure.

The monomer or prepolymer is preferably comprised of single or multiple chemical compositions.

The monomer or prepolymer is preferably a) linear or branched, b) mono-, bi-, tri-, or multi-functional to achieve a pB with linear, branched, hyper-branched, or brush architecture.

The monomer or prepolymer is preferably monofunctional, bi-functional, or multifunctional with reactive or polymerizable functional groups such as amine, hydroxyl, thiol, carboxyl, and vinyl groups among others.

The monomer or prepolymer is preferably hydrophilic, hydrophobic or amphiphilic to achieve the desired pB solubility, physical crosslinking ability, or self-assembly ability.

The monomer or prepolymer is preferably neutral, positively or negatively charged, or combination thereof to achieve a neutral, charged, or zwitterionic pB.

The monomer or prepolymer is preferably polyether, polyester, polyamide, polyacrylate, polyalkyl, polysaccharide, and their derivatives or precursors, as well as the combination thereof.

“PD” as the term is used herein to mean phenolic derivative.
“PDp” as the term is used herein to mean a pB modified with PD.
“Monomer” as the term is used herein to mean non-repeating compound or chemical that is capable of polymerization to form a pB.

“Prepolymer” as the term is used herein to mean an oligomeric compound that is capable of polymerization or polymer chain extension to form a pB. The molecular weight of a prepolymer will be much lower than, on the order of 10% or less of, the molecular weight of the pB.

Monomers and prepolymers can be and often are polymerized together to produce pB.

“pB” as the term is used herein to mean a polymer backbone comprising a polymer, co-polymer, terpolymer, oligomer or multi-mer resulting from the polymerization of pB monomers, pB prepolymers, or a mixture of pB monomers and/or prepolymers. The polymer backbone is preferabley a homopolymer but most preferably a copolymer. The polymer backbone is PDp excluding PD.

“FP” as the term is used herin to mean a polymer backbone functionalized with amine, thiol, carboxyl, hydroxyl, or vinyl groups, which can be used to react with PD to form PDp.
“PD weight percent” as the term is used herein to mean the percentage by weight in PDp that is PD.
“PDP molecular weight” as the term is used herein to mean the sum of the molecular weights of the polymer backbone and the PD attached to said polymer backbone.

In still further embodiments blends of the compounds of the invention described herein, may be prepared with various polymers. Polymers suitable for blending with the compounds of the invention are selected to impart non-covalent interactions with the compound(s), such as hydrophobic-hydrophobic interactions or hydrogen bonding with an oxygen atom on PEG and a substrate surface. These interactions may increase the cohesive properties of the film to a substrate. If a biopolymer is used, it may introduce specific bioactivity to the film, (i.e., biocompatibility, cell binding, immunogenicity, etc.).

Generally, there are four classes of polymers useful as blending agents with the compounds of the invention. Class 1 includes: Hydrophobic polymers (polyesters, PPG) with terminal functional groups (—OH, COOH, etc.), linear PCL-diols (MW 600-2000), branched PCL-triols (MW 900), wherein PCL can be replaced with PLA, PGA, PLAGA, and other polyesters.

Class 2 includes amphiphilic block (di, tri, or multiblock) copolymers of PEG and polyester or PPG, tri-block copolymers of PCL-PEG-PCL (PCL MW=500−3000, PEG MW=500−3000), tri-block copolymers of PLA-PEG-PLA (PCL MW=500−3000, PEG MW=500−3000). In other embodiments, PCL and PLA can be replaced with PGA, PLGA, and other polyesters. Pluronic polymers (triblock, diblock of various MW) and other PEG, PPG block copolymers are also suitable.

Class 3 includes hydrophilic polymers with multiple functional groups (—OH, —NH2, —COOH) along the polymeric backbone. These include, for example, PVA (MW 10,000-100,000), poly acrylates and poly methacrylates, and polyethylene imines.

Class 4 includes biopolymers such as polysaccharides, hyaluronic acid, chitosan, cellulose, or proteins, etc. which contain functional groups.

Abbreviations: PCL=polycaprolactone, PLA=polylactic acid, PGA=Polyglycolic acid, PLGA=a random copolymer of lactic and glycolic acid, PPG=polypropyl glycol, and PVA=polyvinyl alcohol.

It should be understood that the compounds of the invention may be coated multiple times to form bi, tri, etc. layers. The layers may be of the compounds of the invention per se, or of blends of a compound(s) and polymer, or combinations of a compound layer and a blend layer, etc.

Consequently, constructs may also include such layering of the compounds per se, blends thereof, and/or combinations of layers of a compound(s) per se and a blend or blends.

The adhesives of the invention described throughout the specification may be utilized for wound closure and materials of this type are often referred to as tissue sealants or surgical adhesives.

The compounds of the invention may be applied to a suitable substrate surface as a film or coating. Application of the compound(s) to the surface inhibits or reduces the growth of biofilm (bacteria) on the surface relative to an untreated substrate surface. In other embodiments, the compounds of the invention may be employed as an adhesive.

Exemplary applications include, but are not limited to fixation of synthetic (resorbable and non-resorbable) and biological membranes and meshes for hernia repair, void-eliminating adhesive for reduction of post-surgical seroma formation in general and cosmetic surgeries, fixation of synthetic (resorbable and non-resorbable) and biological membranes and meshes for tendon and ligament repair, sealing incisions after ophthalmic surgery, sealing of venous catheter access sites, bacterial barrier for percutaneous devices, as a contraceptive device, a bacterial barrier and/or drug depot for oral surgeries (e.g. tooth extraction, tonsillectomy, cleft palate, etc.), for articular cartilage repair, for antifouling or anti-bacterial adhesion.

In certain embodiments, the present invention provides a polymer of the structure (FIG. 7):

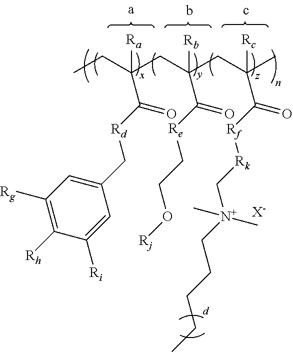

wherein the polymer has mole percentages for each of the monomer components a, b, and c as specified in the table shown in FIG. 8.

One embodiment is a method to reduce microbial fouling on a surface, comprising:

a) providing a surface;

b) functionalizing said surface;

c) providing a phenyl derivative (PD)-poly((meth)acrylic) polymer comprising Formula I:

I

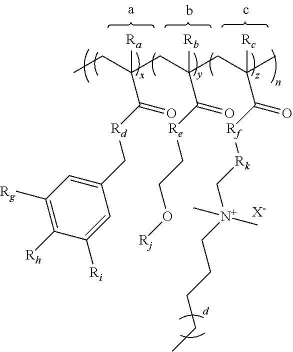

wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;

d) applying an effective amount of said polymer to said functionalized surface;

e) providing silver nitrate;

f) cross-linking said surface and said polymer with said silver nitrate; and

g) reducing said microbial fouling on said surface.

In another embodiment the phenyl derivative is a multihydroxy phenol derivative. In another embodiment, the functionalizing of said surface comprises providing an ammonia plasma, and treating said surface with said ammonia plasma. In another embodiment, the functionalizing comprises creating reactive amine groups on the surface. In another embodiment, the phenyl derivative described above binds to the reactive amine groups.

In another embodiment, the medical device is a urologic device. In still another embodiment, the urologic device is selected from the group consisting of a urinary stent or catheter. In an embodiment, the microbial fouling is bacterial fouling.

Another embodiment is a method of providing a biofouling resistant surface, wherein said method comprises the steps of:

a) providing a medical device surface having been functionalized with reactive amine groups;

b) providing a multihydroxy phenyl derivative (DHPD)-poly(ethylene glycol) polymer comprising Formula I:

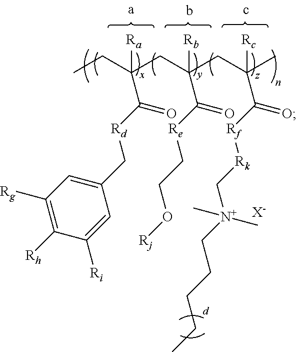

wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;

c) providing an effective amount of silver nitrate: and

d) applying said polymer of Formula I, and said silver nitrate to said surface.

In an embodiment, the polymer of Formula I and said silver nitrate form a coating on said surface.

One embodiment is a biofouling resistant construct, comprising:

a biocompatible surface presenting functional reactive groups; and
a coating comprising the formula:

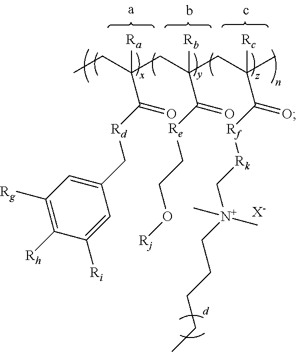

wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;
wherein the molecule of claim 1 is cross-linked and contains an effective amount of silver(0).

In another embodiment there is provided a composition comprising the polymer of the formula:

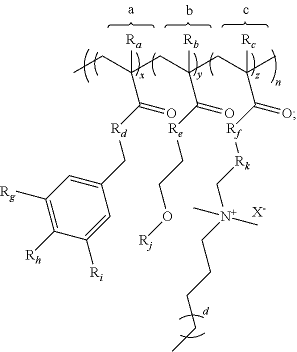

wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;

wherein the polymer is configured to be cross-linked to one of a group consisting of: an adjacent polymer molecule from Formula I, a reactive group on a surface,

wherein the composition further comprises an effective amount of silver(0).

In another embodiment, the composition is crosslinked to a surface treated with ammonia gas plasma.

In another embodiment, there is provided a method for adhering an antibacterial coating to a surface consisting of a PD modified polymer (PDp) according to the formula:

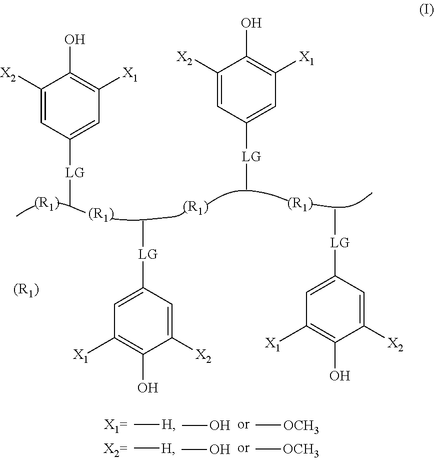

wherein LG is an optional linking group;
PD is a phenolic derivative selected from vanillylamine, 3-methoxytyramine, 3,5-dimethoxy-4-hydroxyphenethylamine, 4-hydroxy-3-methoxy-L-phenylalanine, or tyramine;
R₁ is are monomeric unit which, independently, can be the same or different and is used to form the PDp;
pB is a linear polymeric backbone; and
applying an effective amount of said PDp to at least one surface; and
applying an effective oxidizer to crosslink the PDp.

In an embodiment, the oxidizer is silver. In another embodiment, the PDp consists of multiple monomeric units. In another embodiment, the monomeric units which make up PDp are antibacterial. In another embodiment, the PDp is essentially non-soluble in aqueous solution. In another embodiment, the effective oxidizer is antibacterial. In another embodiment, the PDp-modified linear polymer (PDp) is configured to cure at a predetermined rate, or alternatively to degrade at a predetermined rate, or both.

### BRIEF DESCRIPTION OF THE FIGURES

FIG. 1: General structure of PDp.

FIG. 2: General synthesis scheme 1—Polymerizable PD is copolymerized with polymerizable comonomer to form PDp. P₃ is a polymerizable group such as vinyl, acrylate, or methacrylate group.

FIG. 3: General synthesis scheme 2—Polymer chain extension reaction between a bifunctional prepolymer and a multi-functional chain extender to form a functionalized polymer and the subsequent coupling with PD to form PDp. x, y and Z are functional groups (—NH₂, —OH, —SH, —COOH, etc.), where x reacts only with y, and Z is remained to react with PD.

FIG. 4: General synthesis scheme 3—Reaction of PD with commercially available or prefabricated functionalized polymer to from PDp. Z is a functional group such as —NH₂, —OH, —SH, —COOH, etc., which can react with PD.

FIG. 5. Application of a representative PDp as an adhesive coating (A) and an antifouling coating (B).

FIG. 6. Performance data for polyurethane sheets coated with candidate Surphys polymers, measured changes in contact angle and reduction in E. coli attachment

FIG. 7: Provides the chemical structure of certain embodiments of polymers of the application for surface coating. X=F, Cl, Br or I.

FIG. 8: Provides monomer compositions in mole percent monomer of polymers of the application.

FIG. 9: Shows an exemplary process for coating a device with a multi-functional cross-linked polymer coating.

FIG. 10: Shows an in vitro uropathogen catheter attachment data using embodiments of the present application.

FIG. 11: Shows rabbit urinary infection data using embodiments of the present application.

### REFERENCE TO TABLES

Discussed in the following section is Table 1, which provides the monomer composition for certain polymers of the present application as mole percents of constituent monomers. Table 2 provides the chemical description of certain embodiments used herein. Those tables follow the References section as a group.

### DETAILED DESCRIPTION OF THE INVENTION

In the specification and in the claims, the terms “including” and “comprising” are open-ended terms and should be interpreted to mean “including, but not limited to . . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl , prop-2-yn-1-yl, etc.; butyls such as butan-1-yl, butan-2-yl, 2-methyl-propan-1-yl, 2-methyl-propan-2-yl, cycl obutan-1-yl, but-1-en-1-yl, but-1-en-2-yl, 2-methyl-prop-1-en-1-yl, but-2-en-1-yl , but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term alkoxy (“OR”) includes groups where R is an hydrogen or an alkane chain linked to at least one oxygen.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used.

Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl , prop-1-en-2-yl, prop-2-en-1-yl (allyl), prop-2-en-2-yl, cycloprop-1-en-1-yl; cycloprop-2-en-1-yl ; butenyls such as but-1-en-1-yl, but-1-en-2-yl, 2-methyl-prop-1-en-1-yl, but-2-en-1-yl , but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkyldiyl” by itself or as part of another substituent refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3 -diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3 -diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan- 1,3-diyl, but-1-en-1,1-diyl, but-1-en-1,2-diyl, but-1-en-1,3 -diyl, but-1-en-1,4-diyl, 2-methyl-prop-1-en-1,1-diyl, 2-methanylidene-propan-1,1-diyl, buta-1,3-dien- 1,1-diyl, buta-1,3-dien-1,2-diyl, buta-1,3-dien-1,3-diyl, buta-1,3-dien-1,4-diyl, cyclobut- 1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3 -diyn-1,4-diyl, etc.; and the like. Where specific levels of saturation are intended, the nomenclature alkanyldiyl, alkenyldiyl and/or alkynyldiyl is used. Where it is specifically intended that the two valences are on the same carbon atom, the nomenclature “alkylidene” is used. In other embodiments, the alkyldiyl group comprises from 1 to 6 carbon atoms (C1-C6 alkyldiyl). In another embodiment there are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno,” by itself or as part of another substituent, refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In other embodiments, the alkyleno group is (C1-C6) or (C1-C3) alkyleno. Alternatively, in other embodiment there are provided straight-chain saturated alkano groups, e.g., methano, ethano, propano, butano, and the like.

“Alkylene” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkylene is indicated in square brackets. Typical alkylene groups include, but are not limited to, methylene (methano); ethylenes such as ethano, etheno, ethyno; propylenes such as propano, prop[1]eno, propa[1,2]dieno, prop[1]yno, etc.; butylenes such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In some embodiments, the alkylene group is (C1-C6) or (C1-C3) alkylene. In other embodiments there are provided straight-chain saturated alkano groups, e.g., methano, ethano, propano, butano, and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{b}, ═N—OR^{b}, trihalomethyl, —CF₃, —CN, —OCN, —SCN, —NO, —NO₂, ═N₂, —N₃, —S(O)₂R^{b}, —S(O)₂O⁻, —S(O)_{2OR}^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS (O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C (S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR ^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(_{ORb})(OR^{b}), —C(O)R^{b}, —C(S)R^{b}, —nC(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S) OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

The identifier “PA” refers to a poly(alkylene oxide) or substantially poly(alkylene oxide) and means predominantly or mostly alkyloxide or alkyl ether in composition. This definition contemplates the presence of heteroatoms e.g., N, O, S, P, etc. and of functional groups e.g., —COOH, —NH₂, —SH, or —OH, as well as ethylenic or vinylic unsaturation. It is to be understood any such non-alkyleneoxide structures will only be present in such relative abundance as not to materially reduce, for example, the overall surfactant, non-toxicity, or immune response characteristics, as appropriate, of this polymer. It should also be understood that PAs can include terminal end groups such as PA-O—CH₂—CH₂—NH₂, e.g., PEG-OCH₂—CH₂—NH₂ (as a common form of amine terminated PA). PA-O—CH₂—CH₂—CH₂—NH₂, e.g., PEG—O—CH₂—CH₂—CH₂—NH₂ is also available as well as PA-O—CH₂—CH(CH₃)—O)ₓₓ—CH₂—CH(CH₃)—NH₂, where xx is 0 to about 3, e.g., PEG-O—(CH₂—CH(CH₃)—O)ₓₓ—CH₂—CH(CH₃)—NH₂ and a PA with an acid end-group typically has a structure of PA-O—CH₂—COOH, e.g., PEG-O—CH₂—COOH or PA-O—CH₂—CH₂—COOH, e.g., PEG-O—CH₂—CH₂—COOH. These can be considered “derivatives” of the PA. These are all contemplated as being within the scope of the invention and should not be considered limiting.

Suitable PAs (polyalkylene oxides) include polyethylene oxides (PEOs), polypropylene oxides (PPOs), polyethylene glycols (PEGs) and combinations thereof that are commercially available from SunBio Corporation, JenKem Technology USA, NOF America Corporation or Creative PEGWorks. It should be understood that, for example, polyethylene oxide can be produced by ring opening polymerization of ethylene oxide as is known in the art.

In one embodiment, the PA can be a block copolymer of a PEO and PPO or a PEG or a triblock copolymer of PEO/PPO/PEO.

Suitable MW ranges of the PA's are from about 300 to about 8,000 daltons, 400 to about 5,000 daltons or from about 450 to about 3,500 daltons.

It should be understood that the PA terminal end groups can be functionalized. Typically the end groups are OH, NH₂, COOH, or SH. However, these groups can be converted into a halide (Cl, Br, I), an activated leaving group, such as a tosylate or mesylate, an ester, an acyl halide, N-succinimidyl carbonate, 4-nitrophenyl carbonate, and chloroformate with the leaving group being N-hydroxy succinimide, 4-nitrophenol, and Cl, respectively. etc.

The notation of “L” refers to either a linker or a linking group. A “linker” refers to a moiety that has two points of attachment on either end of the moiety. For example, an alkyl dicarboxylic acid HOOC-alkyl-COOH (e.g., succinic acid) would “link” a terminal end group of a PA (such as a hydroxyl or an amine to form an ester or an amide respectively) with a reactive group of the DHPD (such as an NH₂, OH, or COOH). Suitable linkers include an acyclic hydrocarbon bridge (e.g,, a saturated or unsaturated alkyleno such as methano, ethano, etheno, propano, prop[1]eno, butano, but[1]eno, but[2]eno, buta[1,3]dieno, and the like), a monocyclic or polycyclic hydrocarbon bridge (e.g., [1,2]benzeno, [2,3]naphthaleno, and the like), a monocyclic or polycyclic heteroaryl bridge (e.g., [3,4]furano [2,3]furano, pyridino, thiopheno, piperidino, piperazino, pyrazidino, pyrrolidino, and the like) or combinations of such bridges, dicarbonyl alkylenes, etc. Suitable dicarbonyl alkylenes include, C2 through C15 dicarbonyl alkylenes such as malonic acid, succinic acid, etc.

Additionally, the anhydrides, acid halides and esters of such materials can be used to effect the linking when appropriate and can be considered “activated” dicarbonyl compounds.

Other suitable linkers include moieties that have two different functional groups that can react and link with an end group of a PA. These include groups such as amino acids (glycine, lysine, aspartic acid, etc.), PA's as described herein, poly(ethyleneglycol) bis(carboxymethyl)ethers, polyesters such as polylactides, lactones, polylactones such as polycaprolactone, lactams, polylactams such as polycaprolactam, polyglycolic acid (PGLA), moieties such as tyramine or dopamine and random or block copolymers of 2 or more types of polyesters.

Linkers further include compounds comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR, wherein R is defined above. The term “activated derivative” refers to moieties that make the hydroxyl or amine more susceptible to nucleophilic displacement or for condensation to occur. For example, a hydroxyl group can be esterified by various reagents to provide a more active site for reaction to occur.

A linking group refers to the reaction product of the terminal end moieties of the PA and DHPD (the situation where “b” is 0; no linker present) condense to form an amide, ether, ester, urea, carbonate or urethane linkage depending on the reactive sites on the PA and DHPD. In other words, a direct bond is formed between the PA and DHPD portion of the molecule and no linker is present.

The term “residue” is used to mean that a portion of a first molecule reacts (e.g., condenses or is an addition product via a displacement reaction) with a portion of a second molecule to form, for example, a linking group, such an amide, ether, ester, urea, carbonate or urethane linkage depending on the reactive sites on the PA and DHPD. This can be referred to as “linkage”.

The denotation “DHPD” refers to a multihydroxy phenyl derivative, such as a dihydroxy phenyl derivative, for example, a 3, 4 dihydroxy phenyl moiety. Suitable DHPD derivatives include the formula:

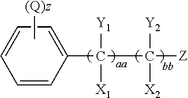

wherein Q is an OH;
“z” is 2 to 5;
each X₁, independently, is H, NH₂, OH, or COOH;
each Y₁, independently, is H, NH₂, OH, or COOH;
each X₂, independently, is H, NH₂, OH, or COOH;
each Y₂, independently, is H, NH₂, OH, or COOH;

### Z is COOH, NH₂, OH or SH;

aa is a value of 0 to about 4;
bb is a value of 0 to about 4; and
optionally provided that when one of the combinations of X₁ and X₂, Y₁ and Y₂, X₁ and Y₂ or
Y₁ and X₂ are absent, then a double bond is formed between the Cₐₐ and C_{bb}, further provided that aa and bb are each at least 1.
In one aspect, z is 3.
In particular, “z” is 2 and the hydroxyls are located at the 3 and 4 positions of the phenyl ring.
In one embodiment, each X₁, X₂, Y₁ and Y₂ are hydrogen atoms, aa is 1, bb is 1 and Z is either COOH or NH₂.

In another embodiment, X₁ and Y₂ are both hydrogen atoms, X₂ is a hydrogen atom, aa is 1, bb is 1, Y₂ is NH₂ and Z is COOH.

In still another embodiment, X₁ and Y₂ are both hydrogen atoms, aa is 1, bb is 0, and Z is COOH or NH₂.

In still another embodiment, aa is 0, bb is 0 and Z is COOH or NH₂.

In still yet another embodiment, z is 3, aa is 0, bb is 0 and Z is COOH or NH₂.

It should be understood that where aa is 0 or bb is 0, then X₁ and Y₁ or X₂ and Y₂, respectively, are not present.

It should be understood, that upon condensation of the DHPD molecule with the PA that a molecule of water, for example, is generated such that a bond is formed as described above (amide, ether, ester, urea, carbonate or urethane).

In particular, DHPD molecules include 3, 4-dihydroxyphenethylamine (dopamine), 3, 4-dihydroxy phenylalanine (DOPA), 3, 4-dihydroxyhydrocinnamic acid, 3, 4-dihydroxyphenyl ethanol, 3, 4 dihydroxyphenylacetic acid, 3, 4 dihydroxyphenylamine, 3, 4-dihydroxybenzoic acid, etc.

In an embodiment, the PD may be a functionalized phenyl derivative, such as a gallate, guaiacolate or catecholate including DHPDs. Some suitable PDs include the formula:

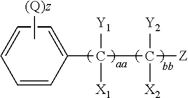

wherein Q is an OH, OR, or an NH2;
“z” is 1 to 5;
R is CH3, an alkane, an alkylene;
each X₁, independently, is H, NH₂, OH, or COOH;
each Y₁, independently, is H, NH₂, OH, or COOH;
each X₂, independently, is H, NH₂, OH, or COOH;
each Y₂, independently, is H, NH₂, OH, or COOH;

### Z is COOH, NH₂, OH or SH;

aa is a value of 0 to about 4;
bb is a value of 0 to about 4; and
optionally provided that when one of the combinations of X₁ and X₂, Y₁ and Y₂, X₁ and Y₂ or Y₁ and X₂ are absent, then a double bond is formed between the Cₐₐ and C_{bb}, further provided that aa and bb are each at least 1.

In one aspect, z is 3.

In another aspect, “z” is 2 and the hydroxyls are located at the 3 and 4 positions of the phenyl ring.

In one embodiment, each X₁, X₂, Yᵢ and Y₂ are hydrogen atoms, aa is 1, bb is 1 and Z is either COOH or NH₂.

In another embodiment, X₁ and Y₂ are both hydrogen atoms, X₂ is a hydrogen atom, aa is 1, bb is 1, Y₂ is NH₂ and Z is COOH.

In still another embodiment, X₁ and Y₂ are both hydrogen atoms, aa is 1, bb is 0, and Z is COOH or NH₂.

In still another embodiment, aa is 0, bb is 0 and Z is COOH or NH₂.

In still yet another embodiment, z is 3, aa is 0, bb is 0 and Z is COOH or NH₂. It should be understood that where aa is 0 or bb is 0, then X₁ and Y₁ or X₂ and Y₂, respectively, are not present.

It should be understood, that upon condensation of either the DHPD or FPD molecule with the PA that a molecule of water, for example, is generated such that a bond is formed as described above (amide, ether, ester, urea, carbonate or urethane).

In particular, DHPD molecules include 3, 4-dihydroxyphenethylamine (dopamine), 3, 4-dihydroxy phenylalanine (DOPA), 3, 4-dihydroxyhydrocinnamic acid, 3, 4-dihydroxyphenyl ethanol, 3, 4 dihydroxyphenylacetic acid, 3, 4 dihydroxyphenylamine, 3, 4-dihydroxybenzoic acid, etc.

### Polymer Synthesis

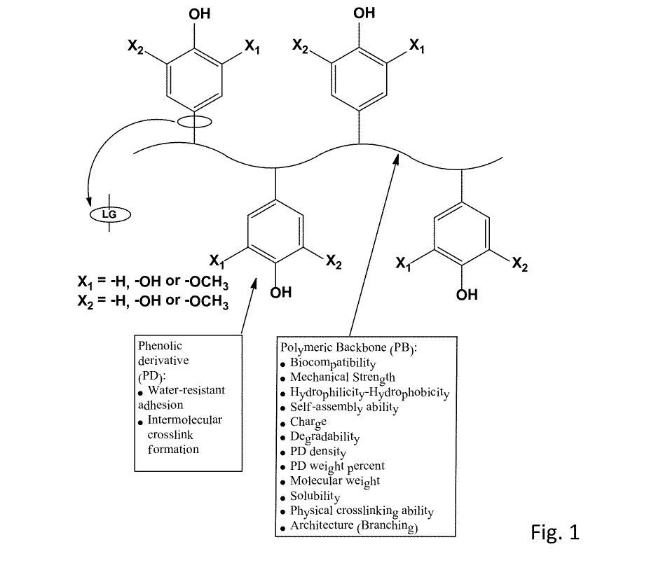

The general structure of the multi-PD adhesive polymer is shown in FIG. 1. This polymer consists of multiple pendant PDs attached to a polymer backbone (pB). PD is incorporated to act as the water-resistant adhesive moiety as well as the intermolecular cross-linking precursor. The number of PDs in a PDp can be used to control the adhesive nature of the polymer, as it has been demonstrated that higher DOPA content correlates to stronger adhesive strengths. Higher PD content can also increase the cure rate of these adhesive polymers.

The polymer backbone can be used to control different physical properties in these multi-PD polymers. A hydrophilic and water-soluble polymer backbone such as poly(ethylene glycol) (PEG) can be used to create a water soluble PDp. Additionally, PEG has a very good biocompatability profile and has been used in many products approved for clinical applications. Hydrophobic segments can be incorporated to increase the stiffness of the polymer backbone, which can result in aggregation of these hydrophobic regions in an aqueous media as well as increasing the mechanical strength of the chemically cured PDp. Different types of chemical linkages can be used to control the stability and the rate of degradation of the polymer backbone. These linkages can vary from stable carbon-carbon, ether, urea, and amide linkages to urethane, ester and carbonate linkages that are easily hydrolysable. Finally, branched polymer backbones can be used to increase the curing rate of PDp.

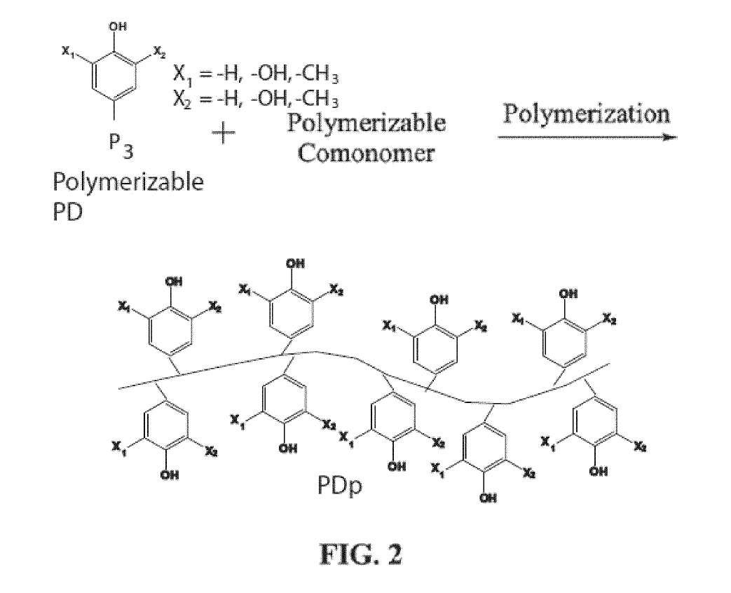

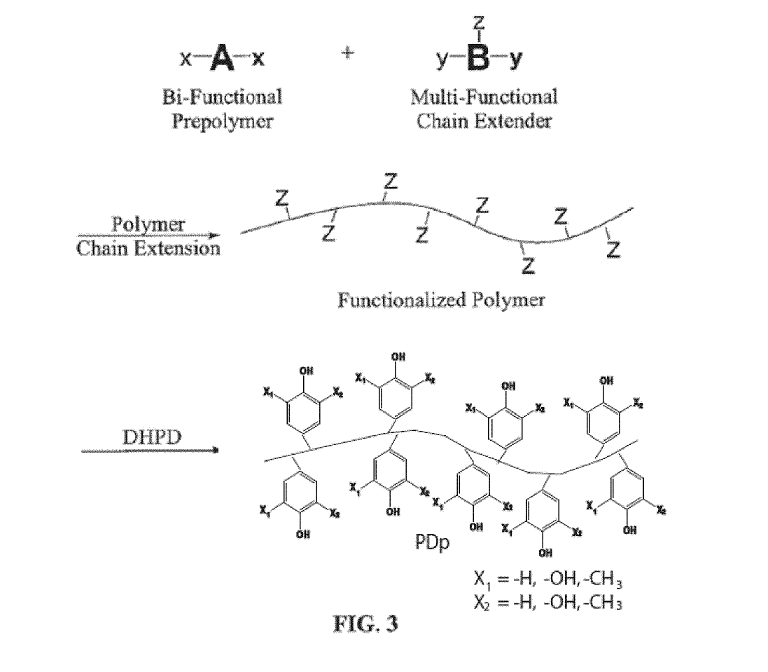

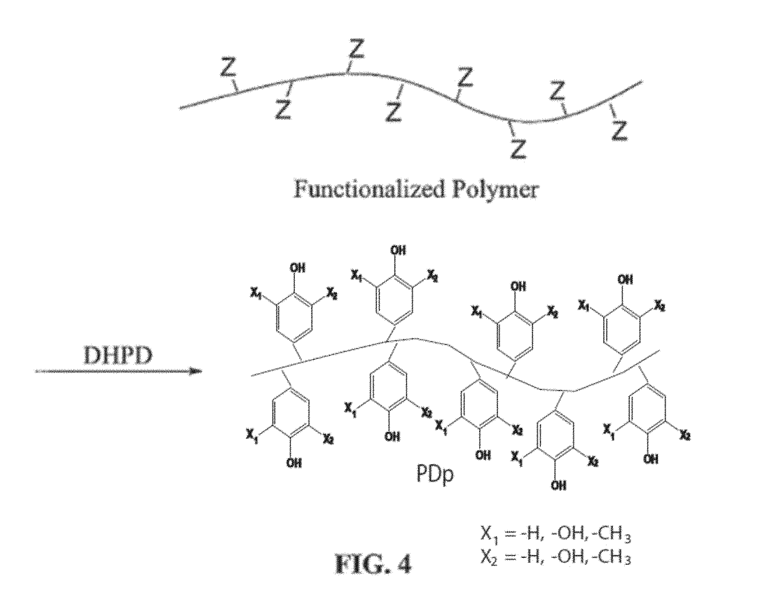

Three general types of synthetic methods were used to create multi-PD adhesive polymers. In the first method (FIG. 2), PD containing a polymerizable group (i.e. vinyl, acrylate, methacrylate) is copolymerized with one or multiple comonomer(s) to form a PDp. In the second method (FIG. 3), a bifunctional prepolymer and a multifunctional chain extender undergo a polymer chain extension reaction to form a functionalized polymer (FP) that carries pendant functional groups (i.e. amine, thiol, hydroxyl, carboxyl, etc.) that can be further modified with PD to form PDp. Finally, a premade FP is reacted with PD to form PDp (FIG. 4). In all three synthesis methods, selection of starting materials (comonomer, prepolymer, FP) can be used to control the physical properties of the polymer backbone and ultimately the PDp.

### Synthetic Method 1

### PD Polymerization

In this section, a series of PDp were created by copolymerizing PD-modified acrylate or methacrylate, acrylamide, methacrylamide (DMA) with one or multiple comonomer(s) using an initiator such as 2,2′-azobis(2-methylpropionitrile) (AIBN). Polymerization was carried out without protection of the reactive PD side chain, which reduces the number of synthetic steps and allows the polymers to be prepared with a higher yield. Although phenolic compounds are known to be inhibitors and radical scavengers, the removal of atmospheric oxygen allowed us to synthesize high molecular weight PDp. Although AIBN-initiated free-radical polymerization is reported here, other polymerization techniques such as atom transfer radical polymerization (ATRP) and reversible addition-fragmentation chain transfer (RAFT) polymerization can potentially be used. However, PD side chain may be required to be protected during polymerization as the metallic catalyst used in ATRP could oxidize PD.

The PDp of various embodiment herein may be linear, random copolymers of DMA and one or more other monomers. Changes can be made to the chemical architecture to further control the physical properties of these adhesive molecules. For example, branching in the polymer backbone can be used to decrease the rate of curing and a branching point can be introduced by using a small amount (<1 mol %) of diacrylated monomers in the polymerization. A larger amount of these bifunctional monomers will result in the formation of a gel network. In addition to branching points, block copolymers can be created using living polymerization methods such as ATRP and RAFT.

### Synthetic Method 2

### Polymer Chain Extension

As shown in FIG. 3, the functionalized polymers (FP) described here are prepared by chain extension of small molecular weight bi-functional prepolymers (x-A-x, MW=200-10,000) with a multifunctional chain extender (y-B(-z)-y). The functionalized polymer is further modified with PD to yield PDp. Since the prepolymer accounts for the majority of the weight fraction (70-95 wt %) of PDp, the composition of this prepolymer will have a significant effect on the physical properties of the PDp. For example, if a hydrophilic prepolymer such as PEG is used, the resulting PDp will be water soluble. Similar water-insoluble PDp can be created using hydrophobic prepolymers such as poly(propylene glycol) or polyesters such as poly(caprolactone) (PCL). More than one type of prepolymer can be used during the chain extension reaction to further refine the physical properties of PDp. Combining hydrophilic and hydrophobic prepolymers will result in a water-soluble PDp that can undergo physical crosslinking in aqueous media, which may result in microscale aggregation of the polymer, increased viscosity, thermally-induced gel formation, or enhancement of mechanical properties of networks chemically cured from PDp. Alternatively, an amphiphilic multi-block copolymer consisting of both hydrophilic and hydrophobic blocks can be used to achieve the same effect. Additionally, incorporation of polyester will render PDp degradable through hydrolysis, and the number of ester linkages in PDp can be used to control the rate of degradation. Finally, the length of the prepolymer can be used to control the density and content of PD, which will affect the adhesive properties as well as the rate of curing of PDp.

In an embodiment, the chain extender consists of a small molecular weight (MW 500 Da) compound that contains two functional groups y that can react with functional groups x on the prepolymer, and at least one functional group Z that can react with PD. The reaction between functional groups x and y results in the formation of ester, amide, urethane, urea, or carbonate linkages between the prepolymer and the chain extender, which leads to the formation of a functionalized polymer. During the chain extension reaction, either x or y needs to be activated for the coupling to occur, which can be done during, or prior to, the reaction.

It may be beneficial for the Z group to be protected, since the functional group may react with either x or y during the polymer chain extension reaction

### Synthetic Method 3

### PD Modification of Functionalized Polymers

In this section, PD is grafted onto pre-made functionalized polymers (FP) that contain pendant functional groups such as —NH₂, —COOH, —OH, or —SH throughout the length of the polymer (FIG. 4). Many different FPs are commercially available and a careful selection should be made based on the desired application of PDp. For example, synthetic FP such as polyvinyl alcohol, polyallylamine, polylysine, and polyacrylic acid exist and are commercially available, but these polymers exhibit poor biocompatibility and none are biodegradable, which make them poor candidates for use as biomaterials. Biopolymers such as proteins or polysaccharides have certain advantages over synthetic polymers (i.e., biocompatibility, biodegradability, bioresorbability, and the ability to interact with native tissue or cells). Protein-based sealants have been approved for clinical use by FDA, which include gelatin—(FloSeal™, Baxter, Inc.), fibrinogen—(Tisseel™, Baxter, Inc.), and bovine serum albumin-based (Bioglue®, Cryolife, Inc.) products. Polysaccharides such as chitosan, alginate, and hyaluronic acid have been studied for various biomedical applications such as cell encapsulation, wound dressing, and cartilage repair. These biopolymers are linear polymers that contain various functional groups that can be modified with PD. Although only modification of gelatin is reported here, other biopolymers with suitable functional groups can be modified with PD using the synthetic path described here.

Gelatin is a protein produced by partial hydrolysis of collagen extracted from the connective tissues of animals such as cows, pigs, and fish. Gelatin contains 10% glutamic acid, 6% aspartic acid, and 4% lysine that can react with PD through amide, ester, or urethane link formation. In an embodiment, water soluble carbodiimide may be used to couple a PD to gelatin (75 Bloom, MW≈22,000). It is contemplated that gelatins may be prepared with a PD content of as much as 8 wt %. It is anticipated that gelatin-based adhesive polymers would be water soluble at concentrations as high as 30 wt % and can undergo physical gelation like unmodified gelatin.

### Applications

It is contemplated that the PDps according to various embodiments described herein may function as 1) tissue adhesives and sealants, 2) adhesive coatings, and 3) antifouling coatings. As a tissue adhesive or sealant , PD in PDp can be used to achieve both cohesive crosslinking and curing of the adhesive as well as interfacial adhesive interaction with both biological and inorganic surface substrates. To function as an adhesive coating, PDp with an elevated PD content may be utilized so that after a portion of the PD was used to attach to the support substrate, there are still unbound PD for binding to a second substrate. For an antifouling adhesive , a relatively low quantity of PD is desired as the majority of an antifouling PDp by weight needs to be constructed of polymers that prevent non-specific adhesion. Depending on the desired applications, PDp were created with different PD contents, physical properties, and chemical compositions.

### Tissue Adhesive and Sealant

To be used as a tissue adhesive or sealant, PDp needs to satisfy a set of stringent criteria. First and most importantly, it should have an adequate safety profile, (i.e., low toxicity, non-immunogenic, non-mutagenic, non-irritating, and non-antigenic) and the bioadhesive should be able to retain its adhesiveness after rigorous sterilization. In the liquid state, the adhesive should have sufficient flow characteristics so that it can be easily applied to the entire wound surface and should be able to displace water from the boundary layer to maximize interfacial interactions. The adhesive must be able to transform from the liquid state into the solid state under mild physiological conditions, and this transition should be rapid to minimize surgery time and to reduce the possibility of infection. After curing, the bioadhesive needs to maintain strong adhesion to different types of tissue in a moist environment while possessing suitable bulk mechanical properties to withstand the different stresses present during functional use. Unlike sutures and other commonly used wound closure materials, adhesives can act as a barrier for tissue growth at the union of the wound edges. Thus, the adhesive must be able to degrade at a rate that approximates the rate of cell growth for satisfactory wound healing, and the degradation products must be nontoxic and capable of being easily reabsorbed or excreted from the body.

Various PDps beneficially undergo a rapid transition from a free flowing liquid to a viscoelastic hydrogel. An aqueous solution of PDp and an equal volume of NaIO₄ solution (0.5 molar equivalent to DHPD) may be mixed using a dual syringe set-up. The amount of time a selected adhesive formulation takes to cure is may be tailored to be under 30 sec, or up to 7 min. Curing time is dependent on such factors as PD content, PDp chemical architecture, and molecular weight. Cohesive crosslinking of DHPDs results in the curing of PDp, thus an elevated PD content is necessary for a fast curing time. Additionally, the rate of curing is also strongly dependent on the chemical structure of the PDp. The brush-like chemical structure of may obstruct pB-bound DMA from making crosslinks efficiently. By providing a short oligomeric linker between PD and a methacrylate group, which allows the PD to be more exposed for crosslink formation rather than buried in a brush of PEG polymers, a shorter cure time would be predicted, on the range of less than 2 minutes.

The various adhesive formulations may function as surgical sealants, such as being used to seal an opening around 3 mm diameter, on a wetted collagen substrate under pressure. ASTM standard F2392 may be followed to determine the burst strength of PDps. Since this experiment tests the ability of a given PDp to bind to a biological substrate in an aqueous environment under stress, the cured adhesives require a good balance of water-resistant adhesive properties as well as bulk mechanical properties. The burst strength of various PDp formulations may range from 5 to 230 mmHg/mm. Various factors, such as adhesive wt %, the polymer backbone chemical structure, and the crosslinking pathway of the PD will have an affect on the burst strength of the adhesive. It is anticipated that the burst strength will increase when the concentration of the polymer was increased, such as from 15 to 30 wt %. This increase may provide improved cohesive properties and crosslinking density in the cured adhesive.

One important criterion for any wound closure material is the ability to biodegrade with time as the wound heals. This is especially important for tissue adhesives and sealants, as a non-degradable material may act as a barrier to the union of wound edges. In vitro degradation analysis of PDp may be performed by submerging the cured adhesives in PBS (pH 7.4) at 37° C. The rate of degradation would likely be dependent on the hydrophilicity of the polymer backbone (pB), since it dictates the rate and the amount of water uptake by the polymer backbone. Thus, various factors such as the synthesis method, the polymer backbone composition, and the prepolymer molecular weight can be used to tailor adhesives with different rates and potentially different modes of degradation.

### Adhesive Coatings

Adhesive-coated tapes, labels, and protective films of all kinds are ubiquitous in everyday life. In the medical field, these adhesive products are used in first-aid bandages, wound dressings, bioelectrodes, transdermal drug delivery patches, and for adhering medical devices to the skin. Good water resistance is needed for these adhesive coatings, both to water applied from outside (i.e. shower), and to water from under the tape or dressing (i.e. perspiration, blood, or wound exudate). Apart from being able to adhere quickly to a biological substrate (i.e., skin), these adhesives also must remain attached to the backing material (i.e., tape or wound dressing backing) so that the adhesive does not transfer onto the skin. Therefore the adhesive should not be water soluble. Although various hydrophobic medical-grade adhesives are available as coatings or films, these lose their ability to adhere to skin when its surface is moistened. Newer generations of adhesives are based on hydrophilic, amphiphilic, or hydrogel-based adhesives, and some of them have demonstrated some level of resistance to moisture. However, the performance of these new adhesives is significantly weakened by high levels of water adsorption or in the presence of water (i.e., showering). Thus a true water-resistant adhesive that can remain adhered to skin during prolonged periods of strenuous exercise and under humid conditions is needed.

### Antifouling Coatings

Unlike the adhesive coatings in the previous section, where the adhesive is designed to adhere to two separate surfaces, polymers for antifouling coating applications are designed to adhere to one surface while preventing other materials from adhering to this surface. For medical devices and implants, preventing proteins, cells, bacteria and other unwanted materials from attaching to the surface of a material is essential in maintaining the desired functionality, longevity, and safety of these devices. Proteins that non-specifically adsorb to material surfaces from extracellular fluids can trigger adverse biological responses, and may interfere with medical device function, as is the case with contact and intraocular lenses, blood-contacting devices, and medical implants and surgical tools. Furthermore, the surfaces of implants, tissue engineering scaffolds, and biosensors functionalized with bioactive ligands (e.g., peptides, proteins and oligonucleotides) benefit from a bioinert background that will not interfere with the desired biological response. Thus, for many biomaterial systems there are tangible benefits to reducing, or eliminating entirely, non-specific interactions between the biomaterial and the fluid or extracellular matrix with which it is in contact.

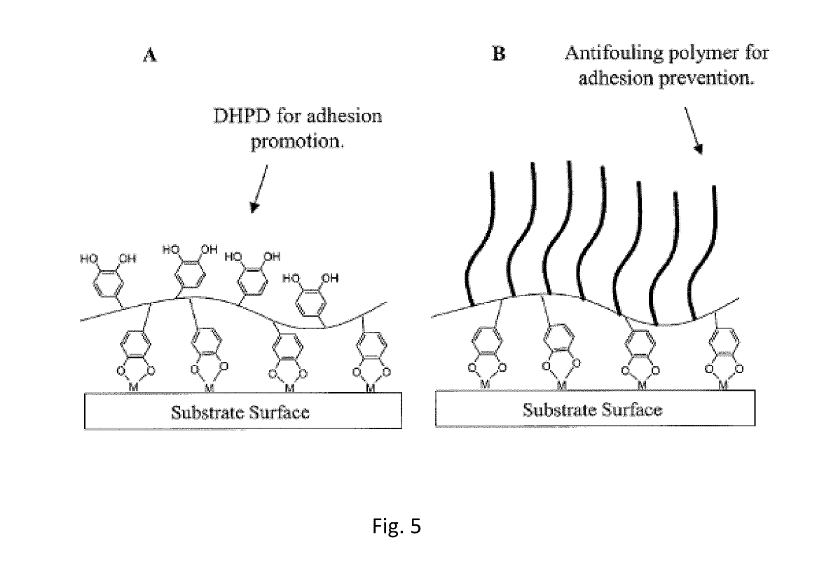

The general design of an antifouling polymer is illustrated in FIG. 5. While the structure in FIG. 5 features a DHPD in the depiction of the general design, it is contemplated that the DHPD may instead be a PD moiety, as has been described previously, and function similarly.

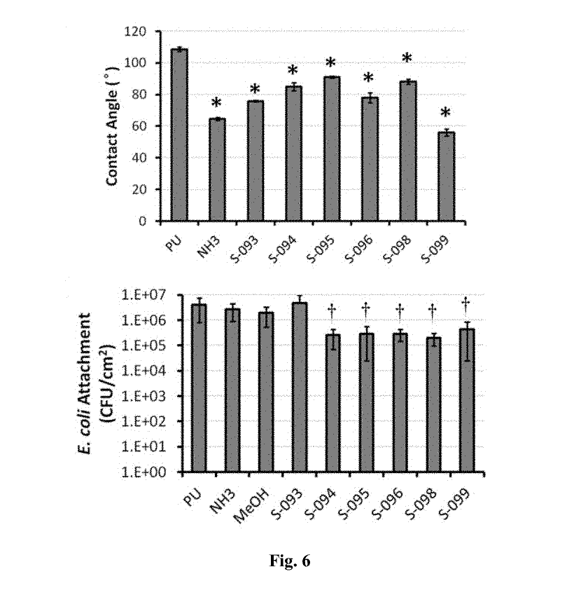

For an effective antifouling application, the polymer requires a relatively small amount of adhesive PD compared to adhesive coatings, while having a large percentage by weight of the polymer with antifouling properties. FIG. 6 summarizes the ability of various PDps to function as antifouling polymers when coated on polyurethane sheets. Surphys coating performance was determined by measuring polyurethane sheets coated with candidate Surphys polymers changes in contact angle and reduction in E. coli attachment (FIG. 6). Ammonia plasma-treated polyurethane sheets were dip-coated with 10 mg/mL Surphys polymer dissolved in methanol and then oxidatively crosslinked using NaIO₄. All candidate Surphys coatings resulted in significant changes in contact angle with polymers containing the highest DMAEMAC₁₂ (Surphys-095 and -098) exhibiting the most pronounced increases in contact angle compared to ammonia plasma treated control. Furthermore, all Surphys coatings, with the exception of S-093, resulted in significant decreases in E. coli attachment compared to untreated polyurethane and ammonia plasma or methanol controls. These results are expected as polymers with increased DMAEMAC₁₂ content are more hydrophobic and have previously been reported to increase bacterial killing.

Advancing water contact angle analysis is a rapid and convenient means of determining if a coating was successfully applied. Advancing contact angles of various hydrophilic PDp-coated surfaces decreased from that of uncoated polyurethane sheets (approximately 110 degrees) down to approximately 58 degrees for the S-099 polymer, signifying that the antifouling coatings were successfully applied to the Polyurethane sheets.

### Antimicrobial Antifouling Coatings

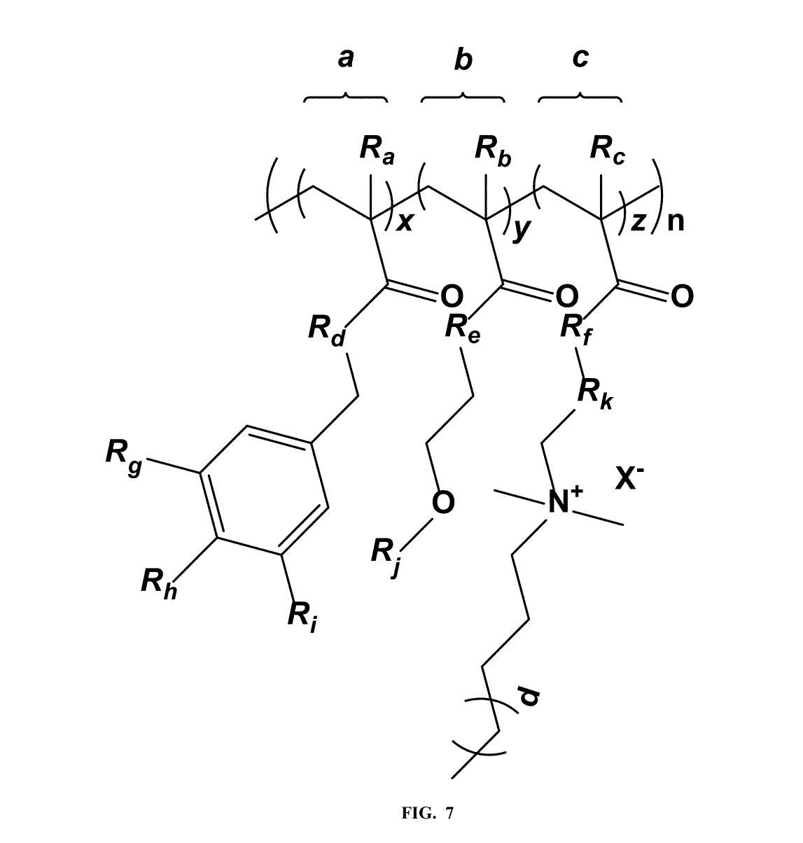

In certain embodiments, antifouling polymers of the present invention comprise antimicrobial properties and compositions. In some embodiments, antifouling polymers of the present invention comprise 3 monomers (monomers a, b, and c as depicted in FIG. 7). In some polymers, a first monomer (a) is selected form a class of monomers comprising a base composition of 3,4-dihydroxyphenyl substituent, for example, DMA, VAMA, DMHPEAMA (Table 2). In further embodiments, a second monomer (b) is selected from a class of monomers comprising AA (Acrylic Acid), HEA, HEMA, MEA (ethylene glycol methyl ether acrylate) (Table 2). In still further embodiments, a third monomer (c) comprises a structure capable of bacterial kill-on-contact. In some embodiments, the MW of the polymer is of 1,000 to 1,000,000 Da, preferably 3,000 to 500,000 Da, more preferably 5,000 to 100,000 Da, and most preferably 15,000 to 70,000. Polymers of the present invention may be prepared by any conventional radical polymerization mechanism including, but not limited to. standard free radical (azo or peroxide initiated), or controlled free radical (ATRP, RAFT, NMRP, CCCT, and the like), as will be discussed.

For polymers prepared under standard free radical conditions, the molecular weight is commonly controlled by the stoichiometry of initiators (e.g. AIBN: Azobisisobutyronitrile, tertiary butylperoctoate, etc.) or through common alkylthiol-containing chain transfer agents (e.g. dodecylmercaptan).

For polymers prepared by controlled free radical processes such as Atom Transfer Radical polymerization (ATRP), Reversible Addition-Fragmentation Chain Transfer (RAFT), Nitroxide-Mediated Radical Polymerization (NMRP), and Cobalt Catalytic Chain Transfer (CCCT), molecular weight and polydispersity is determined by radical equilibria that is controlled by transition metals, thioesters and carbonates, nitroxides, and cobalt complexes respectively.¹

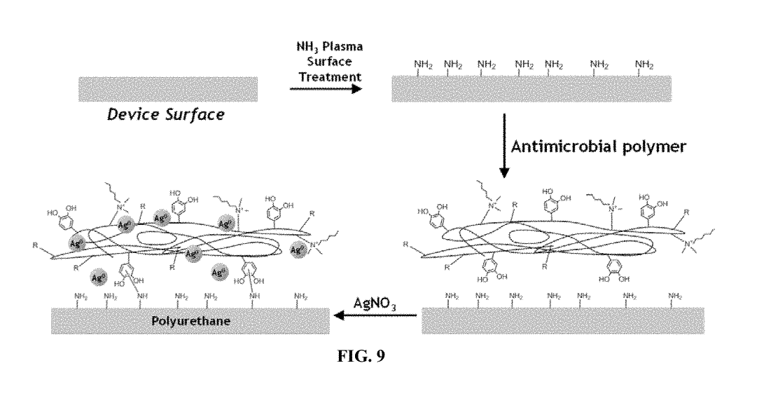

In some embodiments, a surface of a device, for example a polyurethane (PU) surface, is activated with, for example, ammonia (NH₃) plasma gas surface treatment. (FIG. 9) In other embodiments, the surface activated device is contacted with a polymer of the present invention to deposit the polymer on one or more surfaces of the device. In another embodiment, silver nitrate is used for oxidation to promote polymer-polymer cross-linking and covalent attachment of the polymer to surface amine groups of a treated device. In experiments conducted in the development of the present invention, it was further discovered that Ag(0) particles embedded in a coating of the present invention after oxidation with silver nitrate via reduction of ionic to elemental silver Ag⁺→Ag⁰ redox reaction further provide antimicrobial properties to the coating. In other embodiments, crosslinking is accomplished with sodium periodate and/or similar oxidants. Coupling anchoring groups to antifouling polymers significantly reduces bacterial attachment to medical devices. In other embodiments, the antifouling coatings of the present invention prevent bacterial attachment to other types of implantable devices. In further embodiments, the antifouling compounds of the present invention are applied to surfaces in healthcare facilities (e.g., keyboards, elevator buttons, etc.) to prevent the spread of infection.

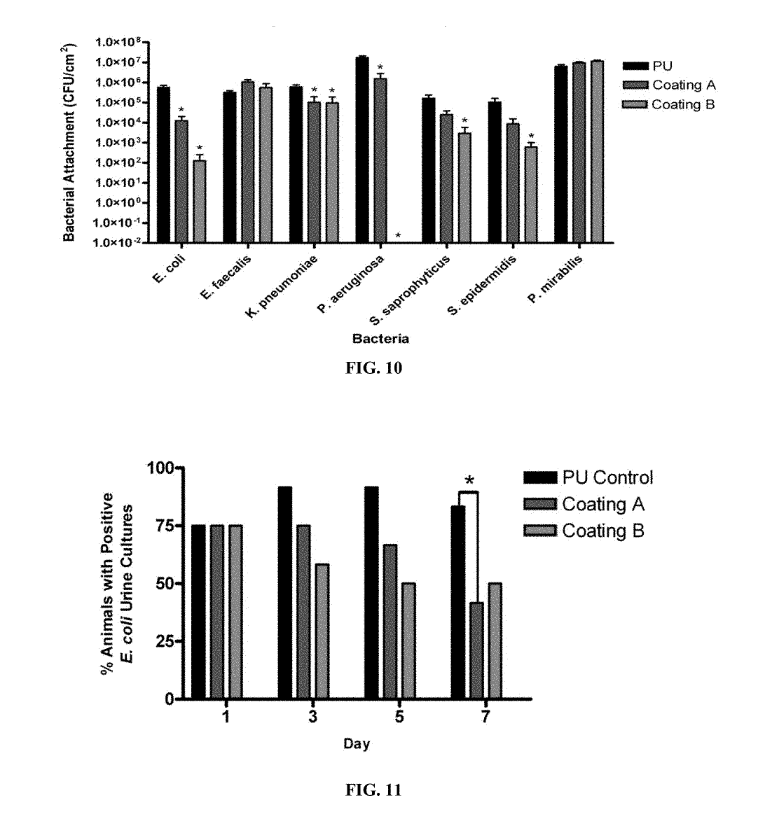

Among the benefits provided by the embodiments described herein is the improved coating performance provided by binding the PDp embodiments depicted in FIG. 7, and providing a functionalized surface for binding the polymer thereon. In an embodiment, the binding surface, e.g. such as that of a catheter or stent, features a plurality of reactive groups to which that PD may bind. This functionalized surface (e.g., having reactive amine groups), following the scheme presented in FIG. 10, will chemically bind to the PDp to anchor the coating to the surface resulting in a functional coating that that remains attached to the surface and less susceptible to leaching, so that the inhibition of bacterial attachment is maximized over a longer period of time. The approach of the present invention is the use of covalent chemical bonds between the PDp and the surface rather than non-covalent interactions such as hydrogen bonding, ionic, metal-oxide, or physical coatings on glass. Moreover the polymer-polymer crosslinking provides a stronger coating and is better able to retain the embedded Ag(0) particles that would otherwise leach out in uncrosslinked coatings Binding of a PDp directly to a functionalized surface with the polymers of FIG. 7 provides a contrast to the binding of the same PDp directly to a surface, lacking the aforementioned functional groups.

### EXAMPLES

### Example 1

### General Route for the Synthesis of Surphys S-093-S-107

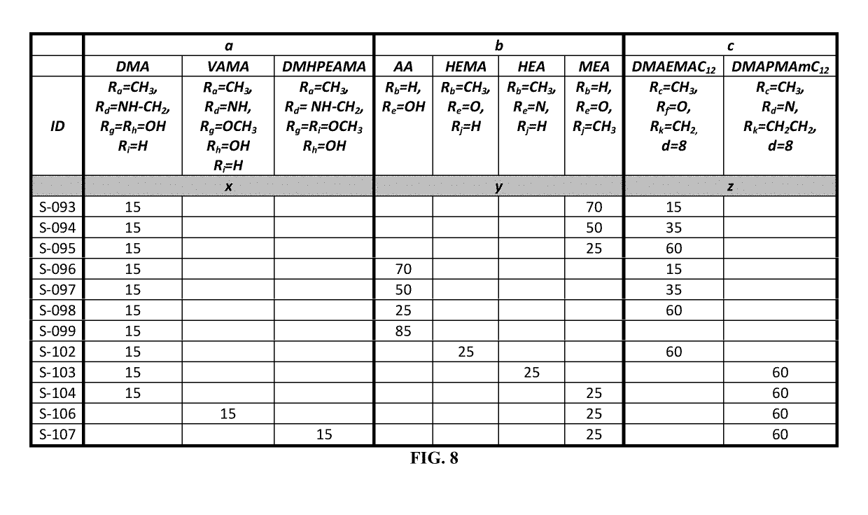

Monomer compositions in mole percent monomers a, b, and c of S-093-S-107 antimicrobial antifouling polymers of the present invention are provided in Table 1 (FIG. 8). For example, for synthesis of S-095 DMA (0.665 g, 3.01 mmol), DMAEMAC₁₂ (4.853 g, 11.94 mmol), MEA (0.659 g, 5.06 mmol), and AIBN (50 mg/(g DMA)) were measured and delivered to an appropriately sized round bottom flask. N,N-Dimethylformamide (DMF) (15 mL/(g DMA)) was added to the reaction vessel. The flask was immediately capped, and an inert gas sparge was applied for a minimum of 20 minutes. The sparge was replaced with an inert gas purge. The round bottom flask was placed in an oil bath preheated to approximately 60° C. It was confirmed that the reaction flask had a pathway to vent. The reaction was allowed to progress overnight. The polymer was precipitated by addition of the reaction solution into diethyl ether (600 mL/(g DMA)). The mixture was placed at −20° C. for a minimum of 1 hour. The polymer was isolated via vacuum filtration and washed 3 times with −20° C. diethyl ether. The polymer was dissolved in a minimal amount of methanol, and precipitated with diethyl ether using a minimal amount of methanol for a rinse. The product was washed with cold diethyl ether three additional times. The product was dried under vacuum overnight. Average Mₙ=46.0 kg/mol (M_{w}=51.2 kg/mol). Average Inherent viscosity (0.1 g polymer/dL in CHCl₃): 0.24 dL/g. Molar composition of molar monomer content was confirmed by ¹H-NMR. Additional polymers were prepared in a shared fashion according to the mole percent of monomers described in Table 1.

For clarity, examples of synthesis for specific molecules and monomers will be presented below.

### Example 2

### Synthesis of Dopamine Methacrylate (DMA)

20 g (238.1 mmol) of sodium bicarbonate and 50.1 g (131.1 mmol) of sodium tetraborate was added to a 1000 mL round bottom flask. 500 mL of nanopure water was added to the round bottom flask which was purged with nitrogen while heating at 50° C. Heating and stirring allowed for the complete dissolution of sodium bicarbonate and sodium tetraborate. The mixture was removed from the heat source and 25 g (131.8 mmol) of dopamine hydrochloride was added and stirred until dissolved. 100 mL of 1N sodium hydroxide was added to the reaction mixture. 23.5 mL (221.4 mmol) of methacrylic anhydride was dissolved in 125 mL of anhydrous THF and added dropwise to the solution over a period of 15 minutes. The reaction was stirred for 21 hours under argon. Once complete, 105 mL of THF was rotary evaporated off. The remaining solution was poured into 1 L of nanopure water. 50 mL of concentrated HCl was added to adjust the pH to ˜0.5. Four extractions with a total of 2 L of ethyl acetate was performed. 1300 mL of ethyl acetate was rotary evaporated off. The remaining solution was added to 1 L of diethyl ether and placed at −15° C. for 45 minutes. The precipitate was suction filtered off and placed under vacuum for 18 hours.

The material was added to 500 mL of nanopure water and stirred for 90 minutes. The insoluble material was suction filtered and placed under vacuum for 2 hours. The material was dissolved in 400 mL of ethyl acetate and 75 mL of methanol through heating. The mixture was allowed to cool to room temperature and 500 mL of diethyl ether was added to the solution which was placed at −15° C. for 90 minutes. The insoluble material was suction filtered off and the remaining solution was poured into 900 mL of heptanes and 300 mL of diethyl ether. The mixture was placed at −15° C. for 2 hours. The precipitate was suction filtered off and washed with diethyl ether. The material was placed under vacuum overnight. 13.83 g of pure material was obtained.

¹H NMR (400 MHz, DMSO/TMS): δ8.73; (s, 1H, —C₆H₃—(OH)₂), 8.62; (s, 1H, —C₆H₃—(OH)₂), 7.92; (s, 1H, —CH₂—C(CH₃)—CONH—CH₂—), 6.61; (d, 1H, —C₆H₃—(OH)₂), 6.56; (s, 1H, —C₆H₃—(OH)₂), 6.41; (d, 1H, —C₆H₃—(OH)₂), 5.60; (s, 1H, CH₂═C(CH₃)—CONH—), 5.28; (s, 1H, CH₂═C(CH₃)—CONH—), 3.22; (m, 2H, —CH₂—CH₂—NHCO—), 2.54; (m, 2H, —CH₂—CH₂—NHCO—), 1.83; (s, 3H, CH₂═C(CH₃)—CONH—).

### Example 3

### Synthesis of DMAEMAC₁₂

75 mL (309.8 mmol) of 1-Bromododecane was added to a 1 L round bottom flask. 210 mL of acetonitrile and 110 mL of chloroform was added to the flask which was purged with argon for 10 minutes. 46 mL (272.4 mmol) of 2-(dimethylamino)ethyl methacrylate was added to the reaction. The reaction was placed at 40-45° C. with argon purging for 20 hours. ˜½ of the solvent was rotary evaporated off. The solution was poured into 1.7 L of diethyl ether and placed at −15° C. for 90 minutes. The precipitate was suction filtered off, washed with MTBE and placed under vacuum overnight. The precipitate was dissolved in 200 mL of chloroform and poured into 1.7 L of diethyl ether. The solution was placed at −15° C. for 3 hours. The precipitate was suction filtered and placed under vacuum until dry. 72.92 g of pure material was obtained.

¹H NMR (400 MHz, DMSO/TMS): δ6.06; (s, 1H, CH₂═C(CH₃)—COO—), 5.75; (s, 1H, CH₂═C(CH₃)—COO—), 4.50; (t, 2H, —COO—CH₂—CH₂—N⁺(CH₃)₂—CH₂—), 3.69; (t, 2H, —COO—CH₂—CH₂—N⁺(CH₃)₂—CH₂—), 3.35; (t, 2H, —COO—CH₂—CH₂—N⁺(CH₃)₂—CH₂—), 3.08; (s, 6H, —COO—CH₂—CH₂—N⁺(CH₃)₂—CH₂—), 1.89; (t, 3H, CH₂═C(CH₃)—COO—), 1.66; (m, 2H, —COO—CH₂—CH₂—N⁺(CH₃)₂—CH₂—CH₂—), 1.24; (m, 2H, —COO—CH₂—CH₂—N⁺(CH₃)₂—CH₂—CH₂—(CH₂)₈—), 0.84; (t, 3H, —COO—CH₂—CH₂—N⁺(CH₃)₂—CH₂—CH₂—(CH₂)₈—CH₃).

### Example 4

### Synthesis of DMAPMAC₁₂

75 mL (309.8 mmol) of 1-Bromododecane was added to a 1 L round bottom flask. 210 mL of acetonitrile and 110 mL of chloroform was added to the flask which was purged with argon for 10 minutes. 50 mL (276.1 mmol) of N-[3-(Dimethylamino)propyl]methacrylamide was added to the reaction. The reaction was placed at 40-45° C. with argon purging for 20 hours. ˜½ of the solvent was rotary evaporated off. The solution was poured into 1.8 L of diethyl ether and placed at −15° C. for 4 hours. The precipitate was decanted off and placed under vacuum overnight. The precipitate was dissolved in 100 mL of chloroform and poured into 3.8 L of diethyl ether. The solution was placed at −15° C. for 6 hours. The precipitate was suction filtered and placed under vacuum until dry. 78.61 g of pure material was obtained.

¹H NMR (400 MHz, DMSO/TMS): δ8.08; (s, 1H, CH₂═C(CH₃)—CONH—), 5.68; (s, 1H, CH₂═C(CH₃)—CONH—), 5.33; (s, 1H, CH₂═C(CH₃)—CONH—), 3.25; (m, 4H, —CONH—CH₂—CH₂—CH₂—N⁺(CH₃)₂—CH₂—), 3.16; (m, 2H, —CONH—CH₂—CH₂—CH₂—N⁺(CH₃)₂—CH₂—), 2.99; (s, 6H, —CONH—CH₂—CH₂—CH₂—N⁺(CH₃)₂—CH₂—), 1.85; (s, 5H, CH₂═C(CH₃)—CONH—CH₂—CH₂—CH₂—N⁺(CH₃)₂—CH₂—CH₂—), 1.60; (s, 2H, CH₂═C(CH₃)—CONH—CH₂—CH₂—CH₂—N⁺(CH₃)₂—), 1.23; (m, 18H, —N⁺(CH₃)₂—CH₂—CH₂—(CH₂)₉—CH₃), 0.85; (m, 3H, —N⁺(CH₃)₂—CH₂—CH₂—(CH₂)₉—CH₃).

### Example 5

### Synthesis of VAMA

14.44 g (172 mmol) of sodium bicarbonate and 50.1 g (94.7 mmol) of sodium tetraborate was added to a 1000 mL round bottom flask. 360 mL of nanopure water was added to the round bottom flask which was purged with nitrogen while heating at 50° C. Heating and stirring allowed for the complete dissolution of sodium bicarbonate and sodium tetraborate. The mixture was removed from the heat source and 18.04 g (95.1 mmol) of vanillylamine hydrochloride was added and stirred until dissolved. 100 mL of 1N sodium hydroxide was added to the reaction mixture. 17 mL (160.2 mmol) of methacrylic anhydride was dissolved in 90 mL of THF and added to the solution. The reaction was stirred for 21 hours under argon. 31 mL of concentrated HCl was added to adjust the pH to ˜2. The reaction was stirred for ˜6 hours. Three extractions with a total of 900 mL of ethyl acetate were performed followed by gravity filtration. 600 mL of ethyl acetate was rotary evaporated off. The remaining solution was added to 3.6 L of MTBE and placed at −15° C. for 24 hours. The precipitate was suction filtered off and placed under vacuum until dry. 9.69 g of pure material was obtained.

¹H NMR (400 MHz, DMSO/TMS): δ8.8; (s, 1H, —C₆H₃—(OH)), 8.34; (s, 1H, —CH₂—C(CH₃)—CONH—CH₂—), 6.82; (s, 1H, —C₆H₃—), 6.68; (d, 1H, —C₆H₃—), 6.63; (d, 1H, —C₆H₃—), 5.67; (s, 1H, CH₂═C(CH₃)—CONH—), 5.33; (s, 1H, CH₂═C(CH₃)—CONH—), 4.20; (d, 2H, —CH₂—NHCO—), 3.72; (s, 3H, —C₆H₃—(OCH₃)), 1.86; (s, 3H, CH₂═C(CH₃)—CONH—).

### Example 6

### Synthesis of 3,5-DM-4-HPEAMA

0.545 g (6.49 mmol) of sodium bicarbonate and 1.39 g (3.57 mmol) of sodium tetraborate was added to a round bottom flask. 15 mL of nanopure water was added to the round bottom flask which was purged with nitrogen while heating at 50° C. Heating and stirring allowed for the complete dissolution of sodium bicarbonate and sodium tetraborate. The mixture was removed from the heat source and 800 mg (3.42 mmol) of 3,5-dimethoxy-4-hydroxyphenethylamine hydrochloride was added and stirred until dissolved. 0.3 mL of 10N sodium hydroxide was added to the reaction mixture. 0.800 mL (5.37 mmol) of methacrylic anhydride was dissolved in 3.25 mL of THF and added to the solution. The reaction was stirred for 16 hours under argon. 1.5 mL of concentrated HCl was added to adjust the pH to ˜0. The reaction was stirred for ˜2 hours. The insoluble material was suction filtered. Two extractions with a total of 100 mL of ethyl acetate were performed. The insoluble material was dissolved into the ethyl acetate extract and washed 3 times with a total of 150 mL of nanopure water. The ethyl acetate fraction was rotary evaporated off. The resulting material was dissolved in 100 mL of acetone, followed by rotary evaporation of the acetone. The material was placed under vacuum to dry for 4 hours. The material was dissolved in 25 mL of methanol and poured into 900 mL of nanopure water. The material was placed at −15° C. for ˜2 hours. No precipitate was observed so the solution was frozen and placed on the freeze drier. The material was dissolved in 20 mL of ethyl acetate with slight heating and placed in the freezer for 1 hour. The resulting precipitate was placed under vacuum until dry. 151 mg of pure material was obtained.

¹H NMR (400 MHz, DMSO/TMS): δ8.07; (s, 1H, —C₆H₂—(OH)), 7.93; (s, 1H, —CH₂—C(CH₃)—CONH—CH₂—), 6.42; (s, 2H, —C₆H₂—), 5.60; (s, 1H, CH₂═C(CH₃)—CONH—), 5.29; (s, 1H, CH₂═C(CH₃)—CONH—), 3.71; (s, 6H, —C₆H₃—(OCH₃)₂), 3.27; (m, 2H, —CH₂—CH₂—NH—CO—), 2.64; (t, 2H, —CH₂—CH₂—NH—CO—), 1.86; (s, 3H, CH₂═C(CH₃)—CONH—).

### Example 7

### Synthesis of Surphys-095

Ethylene glycol methyl ether acrylate was passed through Aluminum Oxide to remove any inhibitor present. 19.441 g (59.5 mmol) of DMAEMAC₁₂, 2.667 g (12.05 mmol) of DMA, 2.605 mL (20.25 mmol) of ethylene glycol methyl ether acrylate (MEA), and 137.6 mg (0.84 mmol) of AIBN were dissolved in 160 mL of DMF. Argon was bubbled through the reaction for 20 minutes. The reaction was then placed at 60-65° C. for 5 hours. The reaction was poured into 1.8 L of diethyl ether and placed at −15° C. for 18 hours. The precipitate was suction filtered off and placed under vacuum for 24 hours. The polymer was then dissolved into 150 mL of methanol and poured into 1.8 L of diethyl ether. This was placed at −15° C. for 18 hours. The precipitate was suction filtered and dried under vacuum for 3 days. The polymer was dissolved in 150 mL of methanol and poured into 1.8 L of diethyl ether and placed at −15° C. for 1 hour. The precipitate was suction filtered and placed under vacuum for 3 days. The polymer was dissolved in 150 mL of methanol and poured into 1.8 L of diethyl ether and placed at −15° C. for 2 hours. The precipitate was suction filtered and placed under vacuum for 5 days. 17.84 g of material was obtained. NMR confirmed <2% monomer was present.

¹H NMR (400 MHz, DMSO/TMS): δ8.66; (s, 2H, —C₆H₃—(OH)₂), 8.25-7.25; (s, 1H, —CONH—CH₂—), 6.61; (s, 2H, —C₆H₃—), 6.38; (s, 1H, —C₆H₃—), 5.0-2.75; (multiple broad peaks), 1.70; (s, 2H, —N⁺(CH₃)₂—CH₂—CH₂—CH₂—), 1.5-0 (multiple broad peaks); Dopamine content based on UV-VIS(DMSO@285 nm): 11.03+/−0.19 Wt %, 0.496+/−0.008 umol DOPA/mg polymer.

### Example 8

### Synthesis of Surphys-102

2-Hydroxyethyl methacrylate (HEMA) was passed through Aluminum Oxide to remove any inhibitor present. 13.085 g (40.0 mmol) of DMAEMAC₁₂, 1.792g (8.1 mmol) of DMA, 1.635 mL (13.48 mmol) of HEMA, and 94.7 mg (0.58 mmol) of AIBN were dissolved in 110 mL of DMF. Argon was bubbled through the reaction for 20 minutes. The reaction was then placed at 60-65° C. for 5 hours. The reaction was poured into 1.8 L of MTBE and placed at −15° C. for 1 hour. The precipitate was suction filtered off and placed under vacuum overnight. The polymer was then dissolved into 100 mL of methanol and poured into 1.8 L of MTBE. This was placed at −15° C. for 2 hours. The precipitate was suction filtered and dried under vacuum for 2 hours. The polymer was dissolved in 100 mL of methanol and poured into 1.8 L of diethyl ether and placed at −15° C. for 2 hours. The precipitate was suction filtered and placed under vacuum overnight. The polymer was dissolved in 150 mL of methanol and poured into 2.8 L of diethyl ether and placed at −15° C. for 2 hours. The precipitate was suction filtered and placed under vacuum for 4 days. 11.86 g of material was obtained. NMR confirmed <2% monomer was present.

¹H NMR (400 MHz, DMSO/TMS): δ8.67; (s, 1H, —C₆H₃—(OH)₂), 8.63; (s, 1H, —C₆H₃—(OH)₂), 8.25-7.25; (s, 1H, —CONH—CH₂—), 6.61; (s, 2H, —C₆H₃—), 6.39; (s, 1H, —C₆H₃—), 5.0-2.75; (multiple broad peaks), 1.69; (s, 2H, —N⁺(CH₃)₂—CH₂—CH₂—CH₂—), 1.5-0 (multiple broad peaks). Dopamine content based on UV-VIS(DMSO@285 nm): 10.02+/−0.06 Wt %, 0.451+/−0.003 umol DOPA/mg polymer.

### Example 9

### Synthesis of Surphys-103

15.16g (44.6 mmol) of DMAPMAC₁₂, 2.005 g (9.06 mmol) of DMA, 1.542 mL (15 mmol) of N-Hydroxyethyl acrylamide (HEA), and 108.8mg (0.66 mmol) of AIBN were dissolved in 120 mL of DMF. Argon was bubbled through the reaction for 20 minutes. The reaction was then placed at 60-65° C. for 5 hours. The reaction was poured into 2.4 L of diethyl ether and placed at −15° C. for 15 hours. The precipitate was suction filtered off and placed under vacuum for 4 hours. The polymer was then dissolved into 75 mL of methanol and poured into 1.9 L of diethyl ether. This was placed at −15° C. for 1 hour. The precipitate was suction filtered and dried under vacuum overnight. The polymer was dissolved in 75 mL of methanol and poured into 1.9 L of diethyl ether and placed at −15° C. for 90 minutes. The precipitate was suction filtered and placed under vacuum for 5 days. The polymer was dissolved in 75 mL of methanol and poured into 900 mL of diethyl ether and placed at −15° C. for 1 hour. The precipitate was suction filtered and placed under vacuum overnight. 12.71 g of material was obtained. NMR confirmed <2% monomer was present.

¹H NMR (400 MHz, DMSO/TMS): δ8.73; (s, 1H, —C₆H₃—(OH)₂), 8.66; (s, 1H, —C₆H₃—(OH)₂), 8.25-7.25; (s, 3H, —CONH-CH₂—), 6.61; (s, 2H, —C₆H₃—), 6.38; (s, 1H, —C₆H₃—), 5.0-2.75; (multiple broad peaks), 1.67; (s, 2H, —N⁺(CH₃)₂—CH₂—CH₂—CH₂—), 1.5-0 (multiple broad peaks). Dopamine content based on UV-VIS(DMSO@285 nm): 9.78+/−0.05 Wt %, 0.44+/−0.002 umol DOPA/mg polymer.

### Example 10

### Synthesis of Surphys-104

20.249 (59.6 mmol) of DMAPMAC₁₂, 2.663g (12.04 mmol) of DMA, 2.605 mL (20.25 mmol) of ethylene glycol methyl ether acrylate (MEA), and 133 mg (0.81 mmol) of AIBN were dissolved in 160 mL of DMF. Argon was bubbled through the reaction for 20 minutes. The reaction was then placed at 60-65° C. for 5 hours. The reaction was poured into 1.8 L of diethyl ether and placed at −15° C. for 90 minutes. The precipitate was suction filtered off and placed under vacuum for 3 days. The polymer was then dissolved into 150 mL of methanol and poured into 1.8 L of diethyl ether. This was placed at −15° C. for 1 hour. The precipitate was suction filtered and dried under vacuum for 11 days. The polymer was dissolved in 150 mL of methanol and poured into 1.8 L of diethyl ether and placed at −15° C. for 90 minutes. The precipitate was suction filtered and placed under vacuum for 2 days. 18.1 g of material was obtained. NMR confirmed <2% monomer was present.

¹H NMR (400 MHz, DMSO/TMS): δ8.70; (s, 1H, —C₆H₃—(OH)₂), 8.63; (s, 1H, —C₆H₃—(OH)₂), 8.25-7.25; (s, 2H, —CONH—CH₂—), 6.59; (s, 2H, —C₆H₃—), 6.36; (s, 1H, —C₆H₃—), 5.0-2.75; (multiple broad peaks), 1.64; (s, 2H, —N⁺(CH₃)₂—CH₂—CH₂—CH₂—), 1.5-0 (multiple broad peaks).

### Example 11

### Synthesis of Surphys-106

9.85 (28.98 mmol) of DMAPMAC₁₂, 1.30 g (5.88 mmol) of VAMA, 2.605 mL (9.87 mmol) of ethylene glycol methyl ether acrylate (MEA), and 73.5 mg (0.45 mmol) of AIBN were dissolved in 78 mL of DMF. Argon was bubbled through the reaction for 20 minutes. The reaction was then placed at 60-65° C. for 5 hours. The reaction was poured into 900 mL of diethyl ether and placed at −15° C. for 60 minutes. The precipitate was suction filtered off and placed under vacuum overnight. The polymer was then dissolved into 75 mL of methanol and poured into 900 mL of diethyl ether. This was placed at −15° C. for 30 minutes. The precipitate was suction filtered and dried under vacuum for 3 hours. The polymer was dissolved in 75 mL of methanol and poured into 900 mL of diethyl ether and placed at −15° C. for 1 hour. The precipitate was suction filtered and placed under vacuum for 5 days. 7.45 g of material was obtained. NMR confirmed <2% monomer was present.

¹H NMR (400 MHz, DMSO/TMS): δ8.76; (s, 1H, —C₆H₃—(OH)), 8.25-7.25; (s, 2H, —CONH—CH₂—), 6.7; (m, 1H, —C₆H₃—), 6.64; (s, 2H, —C₆H₃—), 5.0-2.75; (multiple broad peaks), 1.67; (s, 2H, —N⁺(CH₃)₂—CH₂—CH₂—CH₂—), 1.5-0 (multiple broad peaks).

### Example 12

### Synthesis of Surphys-107

0.998 g (2.94 mmol) of DMAPMAC₁₂, 150 mg (0.54 mmol) of 3,5-DM-4-HPEAMA, 0.128 mL (1 mmol) of ethylene glycol methyl ether acrylate (MEA), and 11 mg (0.07 mmol) of AIBN were dissolved in 8 mL of DMF. Argon was bubbled through the reaction for 20 minutes. The reaction was then placed at 60-65° C. for 5 hours. The reaction was poured into 300 mL of diethyl ether and placed at −15° C. for 30 minutes. The precipitate was suction filtered off and placed under vacuum overnight. The polymer was then dissolved into 23 mL of methanol and poured into 350 mL of diethyl ether. This was placed at −15° C. for 90 minutes. The precipitate was suction filtered and dried under vacuum for 18 hours. The polymer was dissolved in 25 mL of methanol and poured into 350 mL of diethyl ether and placed at −15° C. for 1 hour. The precipitate was suction filtered and placed under vacuum overnight. 500 mg of material was obtained. NMR confirmed <2% monomer was present. ¹H NMR (400 MHz, DMSO/TMS): δ8.10; (s, 1H, —C₆H₂—(OH)), 8.0-7.25; (s, 2H, —CONH—CH₂—), 6.41; (m, 2H, —C₆H₂—), 5.0-2.75; (multiple broad peaks), 1.68; (s, 2H, —N⁺(CH₃)₂—CH₂—CH₂—CH₂—), 1.5-0; (multiple broad peaks).

### Example 13

### PEG Based Monomers which may be Used in this Application

| Monomer | Structure |
|---|---|
| Ethylene glycol methyl ether acrylate | 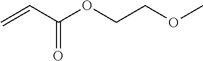 |
| 2-Hydroxyethyl methacrylate | 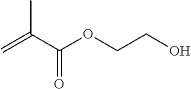 |
| 2-Hydroxyethyl acrylamide | 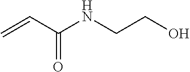 |
| Poly(ethylene glycol) methyl ether methacrylate (Mn~300) | 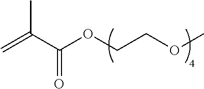 |
| Poly(ethylene glycol) methyl ether methacrylate (Mn~475) | 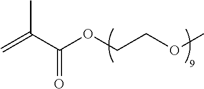 |
| Poly(ethylene glycol) methyl ether methacrylamide (Mn~680) | 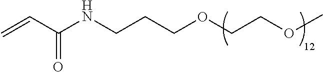 |
| Poly(ethylene glycol) methyl ether methacrylamide (Mn~1085) | 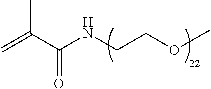 |

### Example 14

### Neutral Hydrophilic Monomers which may be Used in this Application

| Monomer | Structure |
|---|---|
| Acrylamide | 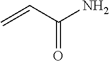 |
| N-Acryloylmorpholine | 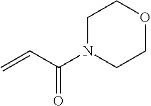 |
| N-Isopropylacrylamide | 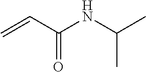 |
| [3- (Methacryloylamino)propyl]dimethyl(3- sulfopropyl)ammonium hydroxide | 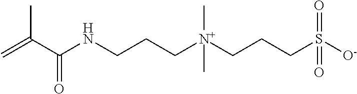 [SMILES: C=C(C)C(=O)NCCC[N+](C)(C)CCCS(=O)(=O)[O-]] |
| 1-Vinyl-2-pyrrolidone | 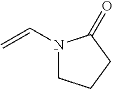 |

### Example 15

### Anionic or Acidic Monomers which may be Used in this Application

| Monomer | Structure |
|---|---|
| 2-Acrylamido-2-methyl- 1-propanesulfonic acid | 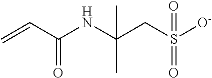 |
| Ethylene glycol methacrylate phosphate | 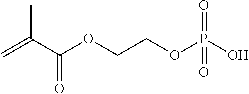 |
| Acrylic acid | 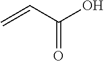 |

### Example 16

### Cationic or Basic Monomers which may be Used in this Application

| Monomer | Structure |
|---|---|
| (3-acrylamido- propyl)trimethyl- ammonium | 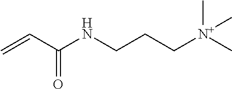 |
| Allylamine | 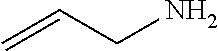 |
| 1,4-diaminobutane methacrylamide | 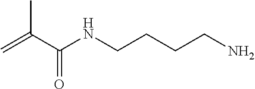 |
| DMAEMAC₁₂ | 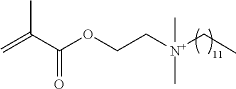 |
| DMAPMAC₁₂ | 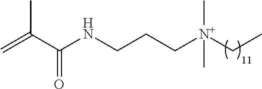 |

### Example17

### Hydrophobic Monomers which may be Used in this Application

| Monomer | Structure |
|---|---|
| 2,2,2-Trifluoroethyl methacrylate | 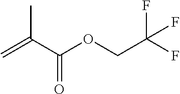 |

### Example 18

### PD Monomers which may be Used in this Application

| Monomer | Structure |
|---|---|
| Vanillylamine methacrylamide | 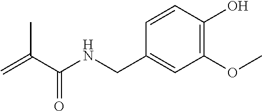 [SMILES: C=C(C)C(=O)NCc1ccc(O)c(OC)c1] |
| 3-methoxytyramine methacrylamide | 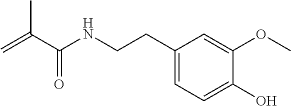 [SMILES: C=C(C)C(=O)NCCc1ccc(O)c(OC)c1] |
| 3,5-dimethoxy-4- hydroxyphenethylamine methacrylamide | 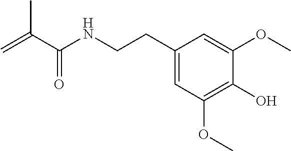 [SMILES: C=C(C)C(=O)NCCc1cc(OC)c(O)c(OC)c1] |
| 3,4-dihydroxyphenethylamine methacrylamide | 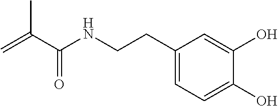 [SMILES: C=C(C)C(=O)NCCc1ccc(O)c(O)c1] |
| 4-hydroxy-3-methoxy-L-phenylalanine methacrylamide | 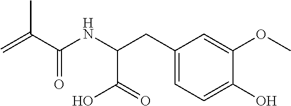 [SMILES: C=C(C)C(=O)NC(Cc1ccc(O)c(OC)c1)C(=O)O] |
| Tyramine methacrylamide | 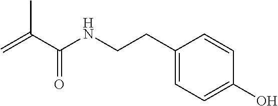 |

Note:
Methacrylamide may be replaced with other vinyl groups

### Example 19

### Catheter Activation and Coating

While the procedure focuses on ammonia gas plasma treatment, it is recognized that in lieu of treatment with ammonia plasma, alternative surface treatments may be utilized for adding functional groups to the surface as may be known to those skilled in the art (e.g. gas cluster ion beam, accelerated neutral atom beam, and common wet chemical treatments).

Prior to plasma treatment, polyurethane samples were cleaned via sequential 10-min sonication in 5% Contrad 70 (Decon Labs Inc., King of Prussia, Pa.) and ultrapure water, and then dried at 55° C. for 4 h. Cleaned samples were placed in a Harrick Plasma Cleaner equipped with a PlasmaFlo gas flow rate controller (Harrick Plasma, Ithaca, N.Y.), then the chamber was pumped down below 100 mTorr and flushed with anhydrous ammonia gas three times.

A polyurethane catheters were treated with ammonia plasma (Ammonia Gas) for 3 minutes at a pressure of 600 mTorr at a power setting of 29.6 W. A 20 mL solution of S-095 in chloroform (Coating A: 2 mg S-095/mL CHCl₃) (low dose), Coating B: 10 mg S-095/mL CHCl₃) (high dose)) was prepared and poured into a dip tube. Plasma treated catheters were slowly dipped into the dip tube and polymer solution, and held for 1 minute. The catheters were removed from the solution and gently shaken to remove excess solution. The catheters were dried for 4 hours in a fume hood. Separately, a 50 mL crosslinking solution of silver nitrate (AgNO₃) in 18 MOhm deionized water at a concentration of a 0.25 mg/mL was prepared. The catheters were placed in a test tube and filled with the silver nitrate crosslinking solution such that the catheters were fully submerged. The solutions were held for 20 hours, removed from the crosslinking solution, rinsed with three portions of 18 MOhm deionized water, and dried at 55° C. for 4 hours. The catheters were packaged and sterilized prior to implantation.

### Example 20

### Reduced In Vitro Uropathogen Attachment Assay

FIG. 10 shows comparative testing of a panel of uropathogens for their ability to adhere to the surface of three catheter surfaces over 24 hours, and to form colony forming units (CFU/cm²) using techniques known to those skilled in the art.

### Example 21

### Reduced In Vivo Urinary Infection

FIG. 11 shows that antimicrobial antifouling compositions and methods of the present invention caused a decrease in the percentage of rabbits with E. coli urinary tract infection. Fewer urinary tract infections were observed in animals with catheters coated with high dose S-095 (n=12) than low dose S-095 (n=12) and uncoated PU alone (n=12). (p <0.05 compared to PU only).

While this invention has been described in conjunction with the various exemplary embodiments outlined above, various alternatives, modifications, variations, improvements and/or substantial equivalents, whether known, or that are or may be presently unforeseen, may become apparent to those having at least ordinary skill in the art. Accordingly, the exemplary embodiments according to this invention, as set forth above, are intended to be illustrative not limiting, various changes may be made without departing from the spirit and scope of the invention. Therefore, the invention is intended to embrace all known or later-developed alternatives, modifications, variations, improvements and/or substantial equivalents of these exemplary embodiments.

TABLE 2
Abbreviations

| Abbreviation | Chemical Description |
|---|---|
| DMA | Dopamine methacrylamide |
| VAMA | Vanillylamine methacyrlamide |
| DMHPEAMA | 3,5-dimethoxy-4-hydroxyphenethylamine |
| | methacyrlamide |
| AA | Acrylic Acid |
| HEMA | Hydroxyethyl methacrylate |
| HEA | N-Hydroxyethyl acrylamide |
| MEA | Ethylene glycol methyl ester acrylate |
| DMAEMAC₁₂ | 2-(dodecyl-dimethylamino)ethyl methacrylate |
| DMAPMAmC₁₂ | 3-(dodecyl-dimethylamino)propyl methacrylamide |

What is claimed is as follows:

1. A method to reduce microbial fouling on a surface, comprising:
a) providing a surface;
b) functionalizing said surface;
c) providing a phenyl derivative (PD)-poly((meth)acrylic) polymer comprising Formula I: 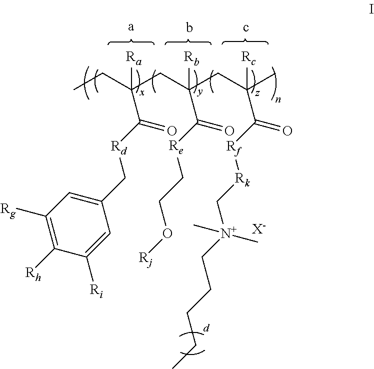
wherein “a” is selected from the group consisting of DMA, VAMA and
DMHPEAMA, “b” is selected from the group consisting of AA, HEMA, HEA and
MEA, and “c” is optionally selected from the group consisting of DMAEMAC₁₂ and DMAPMAmC₁₂;
d) applying an effective amount of said polymer to said functionalized surface;
e) providing silver nitrate;
f) cross-linking said surface and said polymer with said silver nitrate; and
g) reducing said microbial fouling on said surface.

2. The method of claim 1 wherein said phenyl derivative is a multihydroxy phenol derivative.

3. The method of claim 1, wherein the functionalizing said surface comprises providing an ammonia plasma, and treating said surface with said ammonia plasma.

4. The method of claim 3, wherein said functionalizing comprises creating reactive amine groups on said surface.

5. The method of claim 4, wherein said phenyl derivative binds to said reactive amine groups.

6. The method of claim 1, wherein said surface is the surface of a medical device.

7. The method of claim 6, wherein said medical device is a urologic device.

8. The method of claim 7, wherein said urologic device is selected from the group consisting of a urinary stent or catheter.

9. The method of claim 1, wherein said microbial fouling is bacterial fouling.

10. A method of providing a biofouling resistant surface, wherein said method comprises the steps of:
a) providing a medical device surface having been functionalized with reactive amine groups;
b) providing a multihydroxy phenyl derivative (DHPD)-poly(ethylene glycol) polymer comprising Formula I: 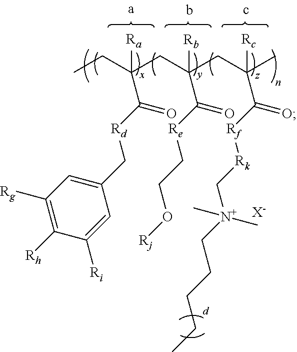
wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;
c) providing an effective amount of silver nitrate: and
d) applying said polymer of Formula I, and said silver nitrate to said surface.

11. The method of claim 10, wherein said polymer of Formula I and said silver nitrate form a coating on said surface.

12. A biofouling resistant construct, comprising:
a biocompatible surface presenting functional reactive groups; and
a coating comprising the formula: 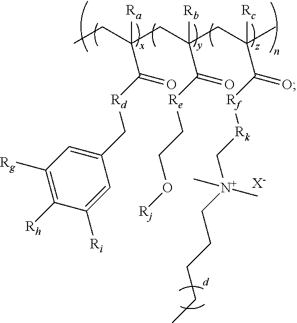
wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;
wherein the molecule of claim 1 is cross-linked and contains an effective amount of silver(0).

13. A composition comprising the polymer of the formula: 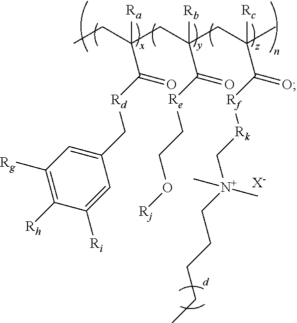
wherein “a” is selected from the group consisting of DMA, VAMA and DMHPEAMA, “b” is selected from the group consisting of AA, HEMA, HEA and MEA, and “c” is optionally selected from the group consisting of DMAEMAC₁₂ and DMAPMAmC₁₂;
wherein the polymer is configured to be cross-linked to one of a group consisting of:
an adjacent polymer molecule from Formula I, a reactive group on a surface, wherein the composition further comprises an effective amount of silver(0).

14. The composition of claim 13, wherein the composition is crosslinked to a surface treated with ammonia gas plasma.

15. A method for adhering an antibacterial coating to a surface consisting of a PD modified polymer (PDp) according to the formula: 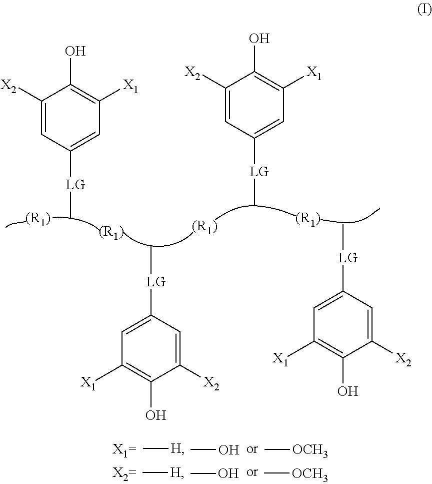
wherein LG is an optional linking group;
PD is a phenolic derivative selected from vanillylamine, 3-methoxytyramine, 3,5-dimethoxy-4-hydroxyphenethylamine, 4-hydroxy-3-methoxy-L-phenylalanine, or tyramine;
R₁ is are monomeric unit which, independently, can be the same or different and is used to form the PDp;
pB is a linear polymeric backbone; and
applying an effective amount of said PDp to at least one surface; and
applying an effective oxidizer to crosslink the PDp.

16. The method of claim 15, wherein said oxidizer is silver.

17. The method of claim 15 where PDp consists of multiple monomeric units.

18. The method of claim 15, wherein the monomeric units which make up PDp are antibacterial.

19. The method of claim 15, wherein the PDp is essentially non-soluble in aqueous solution.

20. The method of claim 15 where the effective oxidizer is antibacterial.

21. The method of claim 15, wherein the PDp-modified linear polymer (PDp) is configured to cure at a predetermined rate.

22. The method of claim 15, wherein the PDp-modified linear polymer (PDp) is configured to degrade at a predetermined rate.
